(19) United States
(12) Patent Application Publication
(10) Pub. No.: US 2003/0180345 A1
(43) Pub. Date: Sep. 25, 2003

(54) Compositions and methods for inhibiting islet dysfunction and autoimmune disorders

(76) Inventors: David J. Hill, London (CA); Brigitte Reusens, Braine-I'Alleud (BE); Claude Remacle, Louvain-la-Neuve (BE)

Correspondence Address: Kenneth I. Kohn, KOHN & ASSOCIATES, PLLC, Suite 410, 30500 Northwestern Highway, Farmington Hills, MI, 48334, US

(21) Appl. No.: 10/364,127

(22) Filed: Feb. 10, 2003

Related U.S. Application Data
(63) Continuation-in-part of application No. PCT/CA01/01137, filed on Aug. 9, 2001.

(30) Foreign Application Priority Data
Aug. 11, 2000 (WO) PCT/CA00/00925

(51) Int. Cl. A61K 31/198; A61K 31/185; A61K 47/00

(52) U.S. Cl. 424/439; 514/553; 514/562

(57) ABSTRACT

The invention relates to a composition comprising an amino acid like structure carrying a sulfur moiety and a biologically acceptable carrier for inhibiting islet dysfunction and/or autoimmune disorders. The structure may be taurine, L-cysteine, L-methionine, or a combination of these. Conditions of islet dysfunction include insulitis, Type 1 diabetes (IDDM), Type 2 diabetes (NIDDM), mature onset diabetes of the young (MODY), and gestational diabetes. Autoimmune disorders include insulitis, Type 1 diabetes, rheumatoid arthritis, thyroiditis and pancreatitis. The composition can act to inhibit islet dysfunction through exerting anti-apoptotic or immunomodulatory activity. Methods are provided for inhibiting islet dysfunction and/or autoimmune disorders by administering an amino acid like structure carrying a sulfur moiety to an individual.

This application is a continuation-in-part of International Patent Application PCT/CA01/01137, which was filed Aug. 9, 2001, and published Feb. 21, 2002, and claims the benefit of priority from International Patent Application PCT/CA00/00925, filed on Aug. 11, 2000, which was published Feb. 21, 2002, the entirety of which is herein incorporated by reference.

### FIELD OF THE INVENTION

The present invention relates to a compositions and methods for inhibiting pancreatic islet dysfunction and for inhibiting autoimmune disorders. The compositions and methods are prophylactic and therapeutically effective against such conditions as insulitis, Type 1 diabetes, and Type 2 diabetes.

### BACKGROUND OF THE INVENTION

Diabetes involves dysfunction of the pancreatic islet cells. In the case of Type 1 diabetes, also referred to as insulin dependent diabetes mellitus (IDDM), dysfunction is initiated in the event of an immunological challenge. In the case of Type 2 diabetes, also referred to as non-insulin dependent diabetes mellitus (NIDDM), islet dysfunction occurs in upon exposure to a homeostatic challenge. Diabetes can alter total β cell mass, as well as the properties of individual β cells.

Type 1 Diabetes and Insulitis. Type 1 diabetes is a chronic autoimmune disease in which insulin-producing cells (β cells) within the pancreatic islets of Langerhans are selectively targeted and destroyed by an infiltrate of immunological cells. This infiltrate causes an inflammatory affect on the islets, known as insulitis.

The development of Type 1 diabetes requires an initial genetic susceptibility, although this susceptibility is insufficient for development of the disease. In susceptible individuals, it has been hypothesized that a triggering event leads to an active autoimmunity attack against β cells, resulting in insulitis, islet β cell dysfunction, diminished insulin secretion, and ultimately, complete β cell destruction. β cells comprise the majority of pancreatic islet cells. Overt Type 1 diabetes onset characterized by hyperglycemia may not be diagnosed until years after an initial triggering event, at which point over 90% of pancreatic β cells are destroyed. When overt diabetes is first recognized, some residual insulin production remains, as demonstrated by the presence of the connecting peptide (C peptide) of proinsulin in the serum. However, the individual usually requires injections of exogenous insulin. Complete β cell destruction is determined when C peptide can no longer be detected in the circulation.

The initiating factor(s) and specific sequence of events leading to Type 1 diabetes, including the relative importance of different cell types and cytokines, are still widely debated. It is generally accepted that insulitis leading to Type 1 diabetes involves cellular migration and infiltration of T lymphocytes, macrophages, and dendritic cells within the pancreatic islets. Immune stimulation of the newly infiltrated cells, and cytokine-regulated effects of such infiltration result in inflammation and β cell destruction (Mandrup-Poulsen, Diabetologia; 1996:39;1005-1029). Interleukin1β (IL 1β), alone or in combination with tumor necrosis factor a (TNFα and interferon γ (IFN γ), exhibits cytotoxicity toward β cells in vitro (Cetkovic et al., Cytokines 1994:6(4):399-406). This cytotoxicity is partly mediated through induction of free radicals such as nitric oxide. (NO), the production of which is catalysed by inducible nitric oxide synthase (iNOS). NO released in β cells leads to nuclear DNA fragmentation and apoptosis, a result which can be partially prevented by iNOS blockers. However, the blockers may not be used in vivo because of the various roles of NO in other organ systems.

Conventional treatment protocols for Type 1 diabetes include immunomodulatory drugs, which merely result in a longer prediabetic period. Other protocols have been suggested which include such immunomodulatory and immunosuppressive agents as levamisol, theophyllin, thymic hormones, ciamexone, antithymocyte globulin, interferon, cyclosporin, nicotinamide, gamma globulin infusion, plasmapheresis or white cell transfusion. Although these protocols may delay onset of Type 1 diabetes, some undesirable side effects are observed. Treatment protocols after onset of Type 1 diabetes are particularly problematic, since by the time diabetes is diagnosed in humans, insulitis has already progressed dramatically, resulting in a β cell loss of more than 80%. Islet transplantation is a potentially successful treatment for Type 1 diabetes, although severe β cell destruction is required to warrant such a procedure.

There is a need for early stage therapies for inhibition of insulitis and other conditions of islet dysfunction. Protocols which could begin prior to disease onset in individuals at risk would be particularly beneficial. Significant progress has been made in identifying risk factors in individuals susceptible to developing Type 1 diabetes. However, the above-noted conventional treatment protocols for Type 1 diabetes are not practical as preventative therapies due to expense and undesirable side-effects. Insulitis is a prediabetic stage, which usually precedes onset of Type 1 diabetes, and thus there is a need for prophylactic protocols for inhibition of insulitis, which could result ultimately in delay or prevention of Type 1 diabetes.

Type 2 Diabetes. Type 2 diabetes often occurs in the face of normal, or even elevated levels of insulin. The condition appears to arise from the inability of tissues to respond appropriately to insulin (i.e. insulin resistance), which challenges the homeostasis of blood glucose. Over time, many individuals with Type 2 diabetes show decreased insulin production and require supplemental insulin to maintain blood glucose control, especially during times of stress or illness.

Conventional treatments for Type 2 diabetes have not changed substantially in many years, and have significant limitations. While physical exercise and a reduction in caloric intake can improve the condition, compliance with such regimens is generally poor. Increasing the plasma level of insulin by administration of sulfonylureas (e.g. tolbutamide, glipizide) to stimulate β cells, or by injection of insulin can result in insulin concentrations that stimulate even highly insulin-resistant tissues. The biguanides increase insulin sensitivity resulting in some correction of hyperglycemia, although some biguanides have side-effects which include lactic acidosis, nausea, or diarrhea.

Accordingly, there exists a continuing need for agents which ameliorate the symptoms of Type 2 diabetes, and especially for those which can prevention or delay onset of Type 2 diabetes or alter susceptibility to Type 2 diabetes later in life.

Sulfur-Containing Amino Acids. Taurine (2-aminoethylsulfonic acid) is a sulfonated β-amino acid, with the sulfonate group present as the acid moiety. Taurine is widely distributed in almost all mammalian tissues. Synthesis of taurine in living organisms can arise via the decarboxylation of cysteic acid and/or via the oxidation of hypotaurine. Both cysteic acid and hypotaurine can be formed from the amino acid cysteine. β-alanine (3-aminopropanoic acid) possesses a structural similarity to taurine with the difference being that the sulfonate group is replaced with a carboxyl group, as shown below. Thus, β-alanine may be used for purposes of comparison with taurine, as a non-sulfur containing control.

Methionine and cysteine are both sulfur-containing α-amino acids. L-Methionine is a non-polar amino acid which is considered “essential” to humans and other animals, such as rats, because it cannot be synthesized by the body an must be derived from dietary sources. L-Cysteine is a polar amino acid which is considered “conditionally essential”, because the body can synthesize L-cysteine from L-methionine. The dietary requirement for L-methionine and L-cysteine is often cited as a combined value. The chemical structures of relevant compounds are provided below, presented in dissociated form.

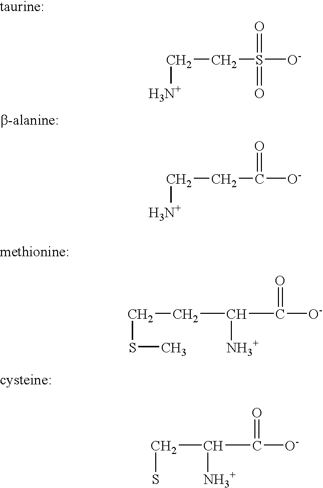

Taurine and/or its analogs have been suggested as a treatment against childhood hyperactivity and learning disabilities in U.S. Pat. No. 4,980,168 to Sahley (Dec. 25, 1990), as a treatment of central nervous system disorders associated with psychotic behavior and dementia, when combined with of neuroleptic drugs in U.S. Pat. No. 5,602,150 to Lidsky (Feb. 11, 1997), and as a general use nutritional supplement in U.S. Pat. No. 4,751,085 to Gaull (Jun. 14, 1988).

Early investigations into dietary supplementation of taurine in the streptozotocin-induced hyperglycemic mouse model found a reduction in hyperglycemia with taurine supplementation (Tokunaga et al., Biochem Pharmacol 1979; 28:2807-2811). The investigators attributed this effect to action of taurine on the cell membrane. A low protein maternal diet has been shown to reduce serum taurine levels and fetal islet insulin secretion (Cherif, et al. J Endocrinology 1996;151:501-506), an effect which can be ameliorated by taurine supplementation (Cherif et al., J. Endocrinology 1998; 159: 341-348). However, this observation merely considered insulin release alone, with no regard for such parameters as islet survival or number. Nakaya et al. found that taurine supplementation improved insulin sensitivity in a rat model of insulin resistance and type II diabetes (Am J Clinical Nutrition 2000: 71(1):54-58). The investigators attributed this effect to reduced hepatic and serum cholesterol and triglyceride levels in the taurine-supplemented group, since taurine encouraged cholesterol conversion to bile acid.

Maternal Nutrition and β Cell Ontogeny. Normal islet β cell ontogeny involves a turnover of cells as a result of a balance of cell replication, islet neogenesis and programmed cell death. This ontogeny is influenced by nutrition in the fetus and neonate. Inadequate nutrition at early developmental stages may yield an adult population of β cells which are inappropriately responsive to metabolic (homeostatic) or immunological challenge. Specifically, a low protein diet may induce long-term changes in proliferative cell cycle kinetics and rates of developmental apoptosis of the β cells either during fetal life, neonatal development, or both.

After birth, β cells undergo a deletion of cells by apoptosis and are replaced through β cell replication and islet neogenesis. In the rat fetus, the cellular area immunostained for insulin increases 2-fold over 2 days just prior to term, due to both β cell replication, and recruitment and maturation of undifferentiated β cell precursors. In mouse embryos dorsal and ventral pancreatic buds appear at embryonic day E9.5 from mid-gut endoderm, and fuse by day E16-17. Each bud forms highly branched structures and the acini and ducts are distinguishable at day E14.5, with amylase being detectable in acinar tissue. Endocrine cells appear early in bud development and represent 10% of the pancreas by day E15.5, initially existing as individual cells or small clusters close to the pancreatic ducts. These endocrine cells form mature islets, with outer αcells and an inner mass of β, D, and PP cells, a few days before birth. A similar appearance is observed by early third trimester in the human. The growth and cytodifferentiation of the pancreas depends on mesenchymal-epithelial interactions. Pancreatic mesenchyme accumulates around the dorsal gut epithelium and induces pancreatic bud formation and branching.

Neogenesis of islets is rapid in the fetus, continues through neonatal life in the rat, but ceases shortly after weaning. This derives not only from β cell replication but from the recruitment and maturation of undifferentiated β cell precursors (Kaung, Dev. Dyn. 1994; 200: 163-175). In the adult, the number of pancreatic β cells undergoing mitosis is substantially reduced, thereby decreasing β cell replication in the adult relative to the fetus. A change to an adult phenotype of non-proliferative β cells is precipitated by a transient wave of apoptosis occurring in islets from neonatal rats at about 7 to 14 days of age, also referred to as developmental β cell apoptosis (Petrik et al., Endocrinology 1998;139: 2994-3004). The number of apoptotic cells within rat islets increases 3-fold by 14 days of age, relative to the number at either 4 or 21 days. During developmental β cell apoptosis, islet β cells contain increased levels of immunoreactive inducible nitric oxide synthase (iNOS), suggesting that endogenous levels of NO within islets may be functionally linked to this transient wave of apoptosis. A similar wave of β cell apoptosis occurs in the human fetus during third trimester.

β cell mass is not altered appreciably at the time of developmental β cell apoptosis, suggesting that a new population of β cells compensates for those lost by apoptosis. Increased numbers of insulin-positive cells are seen near to the ductal epithelia after 12 days, suggesting that the new generation of islet cells maintain β cell mass. Partial replacement of β cells at the neonatal stage provides a cell population having improved metabolic control in later adult life. Aberrant developmental apoptotic deletion of fetal-type cells or neogenesis of adult-type islets hinders the ability of an individual to deal with autoimmune or homeostatic metabolic stress in later life.

Intrauterine and neonatal growth abnormalities caused by restriction or alteration of nutritional metabolites alter β cell mass. In a rigorous analysis in which maternal calorie intake was reduced by 50% from day 15 of gestation until term, it was shown that β cell mass was reduced in the newborn rat, due to a reduction in the number of islets (Garofano et al. Diabetologia 1997; 40: 1231-1234). If a normal diet is restored at birth, the β cell mass returns to that of controls by weaning. However, if energy restriction continues during neonatal life, irreversible changes in β cell mass result.

Intrauterine growth retardation (IUGR) in humans and rats is associated with a reduced pancreatic β cell number at birth, and is a major risk factor for Type 2 diabetes, hyperlipidemia, and hypertension in later life. Impaired glucose tolerance can be detected as early as 7 years of age in children having a low birth weight who are thin. Perturbations of prenatal or neonatal nutrition lead to altered β cell ontogeny, and result in a population of β cells qualitatively ill-suited to subsequently survive metabolic or immunological stresses. There is a need for strategies for intervention in IUGR to reduce the risk of later development of Type 2 diabetes.

A low protein diet model shows a strong effect of nutritional deficiency on fetal islet development, and illustrates that the neonatal period is a time of islet plasticity which will have life-long consequences for glucose homeostasis. Protein restriction in an otherwise isocaloric diet provides a useful model of malnutrition, given the major role of amino acids as insulin secretagogues for the fetal islets, and considering that glucose responsiveness develops shortly before birth. Intrauterine malnutrition, manifest as protein deficiency, can induce alterations in the development of the fetal endocrine pancreas. A low protein diet given to pregnant rats decreased islet cell proliferation and pancreatic insulin content in offspring (Snoeck et al, Biol. Neonate 1990: 57; 107-118) and insulin release in offspring (Dahri et al., Diabetes 1991:40; suppl. 2;115-120). Maternal supplementation of taurine in a protein deficient diet was shown to preserve the fractional release of insulin from fetal islets of the offspring, as compared with offspring of animals fed a control diet (Cherif et al., J Endocrinology 1998; 159: 341-348).

There is a need to counteract the damage to the fetus which can be induced by poor maternal nutrition. Further, there is a need to optimize nutrition for pregnant individuals, and individuals known to have genetic susceptibility or other risk factors that predispose the individual or their offspring to conditions of islet dysfunction, in particular insulitis, Type 1 diabetes and Type 2 diabetes.

### SUMMARY OF THE INVENTION

It has surprisingly been found that amino acid like structures carrying a sulfur moiety alter the tendency of a susceptible individual to develop conditions of islet dysfunction. Further, it has been found that maternal supplementation of amino acid like structures carrying a sulfur moiety inhibits islet dysfunction in offspring. The prior art observations of the effect of taurine on insulin secretion do not suggest or infer any effect of amino acid like structures carrying a sulfur moiety on islet dysfunction.

It is an object of the invention to provide a composition and method for inhibiting islet dysfunction which obviate or mitigate one or more of the above-noted deficiencies in the prior art. A further object of the invention is to provide a composition and method for maternal supplementation which inhibits islet dysfunction in offspring.

Thus, according to the invention, there is provided a composition for inhibiting islet dysfunction. The composition comprises an amino acid like structure carrying a sulfur moiety and a biologically acceptable carrier. The amino acid like structure carrying a sulfur moiety may be, for example, taurine, L-cysteine, L-methionine, or a combination thereof. According to the invention, islet dysfunction may be manifest in such conditions as insulitis, Type 1 diabetes, Type 2 diabetes, mature onset diabetes of the young (MODY), or gestational diabetes. The composition may be used to inhibit islet dysfunction in the offspring of a pregnant mammal, and thus may be formulated as a maternal supplement. Further, the composition may be used to inhibit islet dysfunction in the suckling offspring of a lactating mammal. Thus the invention further provides an infant formula comprising an amino acid like structure carrying a sulfur moiety, for example a sulfur-containing amino acid, for inhibition of islet dysfunction in an infant.

Further, according to the invention, there is provided a method of inhibiting islet dysfunction comprising administration of an effective amount of an amino acid like structure carrying a sulfur moiety to a mammal in need thereof. According to one embodiment of this method, the amino acid like structure carrying a sulfur moiety may be taurine, L-cysteine, L-methionine, or a combination thereof. This method may be implemented for inhibiting islet dysfunction in the offspring of a pregnant mammal by administering an effective amount of the amino acid like structure carrying a sulfur moiety to the pregnant mammal. The method may also be implemented for inhibiting islet dysfunction in the suckling offspring of a lactating mammal.

Additionally, the invention provides the use of an effective amount of an amino acid like structure carrying a sulfur moiety for preparation of a medicament for inhibiting islet dysfunction in a mammal. Further, the invention relates to a commercial package comprising an effective amount of an amino acid like structure carrying a sulfur moiety together with instructions for use in inhibiting islet dysfunction.

The invention also relates to the use of an effective amount of an amino acid like structure carrying a sulfur moiety for inhibition of islet dysfunction in a mammal in need thereof. According to one embodiment of this use, the amino acid like structure carrying a sulfur moiety may be taurine, L-cysteine, L-methionine, or combinations thereof. This use may be implemented for inhibiting islet dysfunction in the offspring of a pregnant mammal by delivery of the amino acid like structure carrying a sulfur moiety to the pregnant mammal. Further, the use according to the invention may be implemented for inhibiting islet dysfunction in the suckling offspring of a lactating mammal, or for delivery to an infant via an infant formula.

It has also been found that amino acid like structures carrying a sulfur moiety alter the tendency of susceptible individuals to develop autoimmune disorders. Further, it has been found that maternal supplementation of amino acid like structures carrying a sulfur moiety inhibits autoimmune disorders in offspring. The prior art observations of the effect of taurine on insulin secretion do not suggest or infer any effect of amino acid like structures carrying a sulfur moiety on autoimmune disorders.

It is a further object of the invention to provide a composition and method for inhibiting autoimmune disorders which obviate or mitigate one or more of the above-noted deficiencies in the prior art. A further object of the invention is to provide a composition and method for maternal supplementation which inhibits autoimmune disorders in offspring.

Thus, according to the invention, there is provided a composition for inhibiting autoimmune disorders. The composition comprises an amino acid like structure carrying a sulfur moiety and a biologically acceptable carrier. The amino acid like structure carrying a sulfur moiety may be, for example, taurine, L-cysteine, L-methionine, or a combination thereof. According to the invention, an autoimmune disorder may be manifest in such conditions as insulitis, Type 1 diabetes, rheumatoid arthritis, thyroiditis, and pancreatitis. The composition may be used to inhibit autoimmune disorders in the offspring of a pregnant mammal, and thus may be formulated as a maternal supplement. Further, the composition may be used to inhibit autoimmune disorders in the suckling offspring of a lactating mammal. Thus the invention further provides an infant formula comprising an amino acid like structure carrying a sulfur moiety, for example a sulfur-containing amino acid, for inhibition of autoimmune disorders in an infant.

Further, according to the invention, there is provided a method of inhibiting autoimmune disorders comprising administration of an effective amount of an amino acid like structure carrying a sulfur moiety to a mammal in need thereof. According to one embodiment of this method, the amino acid like structure carrying a sulfur moiety may be taurine, L-cysteine, L-methionine, or a combination thereof. This method may be implemented for inhibiting autoimmune disorders in the offspring of a pregnant mammal by administering an effective amount of the amino acid like structure carrying a sulfur moiety to the pregnant mammal. The method may also be implemented for inhibiting autoimmune disorders in the suckling offspring of a lactating mammal.

Additionally, the invention provides the use of an effective amount of an amino acid like structure carrying a sulfur moiety for preparation of a medicament for inhibiting autoimmune disorders in a mammal. Further, the invention relates to a commercial package comprising an effective amount of an amino acid like structure carrying a sulfur moiety together with instructions for use in inhibiting autoimmune disorders.

The invention also relates to the use of an effective amount of an amino acid like structure carrying a sulfur moiety for inhibition of autoimmune disorders in a mammal in need thereof. According to one embodiment of this use, the amino acid like structure carrying a sulfur moiety may be taurine, L-cysteine, L-methionine, or combinations thereof. This use may be implemented for inhibiting autoimmune disorders in the offspring of a pregnant mammal by delivery of the amino acid like structure carrying a sulfur moiety to the pregnant mammal. Further, the use according to the invention may be implemented for inhibiting autoimmune disorders in the suckling offspring of a lactating mammal, or for delivery to an infant via an infant formula.

### BRIEF DESCRIPTION OF THE DRAWINGS

Preferred embodiments of the present invention will now be described, by way of example only, with reference to the attached Figures.

FIG. 1 illustrates the effect of a maternal control (C) versus low protein (LP) diet on fetal islet cell apoptosis induced by sodium nitropruside (SNP) at 0, 10 or 100 μmol/l, as quantified by confocal microscopy.

FIG. 2A, FIG. 2B and FIG. 2C show the effect of taurine, L-methionine and β-alanine, respectively, on SNP-induced apoptosis of fetal islet β cells derived from animals exposed to a maternal control (C) versus low protein (LP) diet, as quantified by confocal microscopy.

FIG. 3 illustrates the effect of taurine (0, 0.3, or 3 mmol/l) on the in vitro mortality of fetal β cells induced by SNP (100 μmol/l), as quantified by confocal microscopy.

FIG. 4 demonstrates quenching of peroxynitrite formation in vitro by fetal islet cells in the presence of taurine (0.3 or 3 mmol/l), L-methionine (0.1 or 1 mmol/l) or β-alanine (0.3 or 3 mmol/l). Quenching is illustrated by a decrease in chemiluminescent light intensity.

FIG. 5 illustrates IL1β-induced apoptosis in cultured fetal islets from animals exposed to a maternal control (C) or low protein (LP) diet, and the protective effect of taurine (0, 0.3 or 3 mmol/l) against IL1β-induced apoptosis as quantified by confocal microscopy.

FIG. 6 illustrates the effect of in vitro taurine (0, 1.25, or 2.5 mmol/l) on the proliferation rate of fetal islet cells derived from animals exposed to a maternal control (C) or low protein (LP) diet. Proliferation was quantified using bromodeoxyuridine (BrdU) incorporation.

FIG. 7 illustrates the effect of a maternal control (C) or low protein (LP) diet with and without taurine supplementation on islet cell proliferation at four developmental stages: fetal day 21.5 (F21.5), and post-natal days 12 (PN 12), 14 (PN 14) and 30 (PN 30), quantified as the percentage of cells testing immunopositive for BrdU incorporation.

FIG. 8 illustrates the effect of a maternal control (C) or low protein (LP) diet with and without taurine supplementation on islet cell apoptosis at fetal day 21.5 (F21.5), and post-natal days 12 (PN 12), 14 (PN 14) and 30 (PN 30).

FIG. 9 illustrates the effect of a maternal control (C) or low protein (LP) diet with and without taurine supplementation on IGF-II levels in islet cells isolated at fetal day 21.5 (F21.5), and post-natal days 12 (PN 12), 14 (PN 14) and 30 (PN 30).

FIG. 10A to FIG. 10D show the effect of a maternal control (C) or low protein (LP) diet with and without taurine supplementation on Fas, Fas ligand, iNOS, and pancreatic VEGF, respectively, in islet cells isolated at fetal day 21.5 (F21.5), and post-natal days 12 (PN 12), 14 (PN 14) and 30 (PN 30), as quantified using immunoreactivity.

FIG. 11A and FIG. 11B illustrate the effect of a maternal control (C) or low protein (LP) diet with and without taurine supplementation (+T) on vascular density and vessel numbers per unit area, respectively.

FIG. 12A to FIG. 12D show the influence of four maternal diet treatments: control (C), control+taurine (C+Taurine), low protein (LP), and low protein+taurine (LP+Taurine), respectively, on islet cell apoptosis under in vitro conditions including taurine supplementation (0, 0.3 and 3.0 mmol/l), in the presence or absence of SNP (100 μmol/l) or IL-1β (50 U/ml).

FIG. 13 illustrates the influence of dietary taurine supplementation on incidence of insulitis in NOD mice at 12 weeks of age. Diet treatments were control (C) or taurine supplemented (C+Taurine). Incidence of insulitis was determined histologically.

FIG. 14 illustrates the severity of insulitis within individual islets from female NOD Mice exhibiting insulitis at 12 weeks of age. Within an animal, islets not illustrating insulitis were not scored for severity. Each islet showing insulitis was scored as either slight, medium or heavy, and the percent of total islets in each category is shown. Diet treatments were control (C) or taurine supplemented (C+Taurine).

FIG. 15 illustrates the effect of maternal taurine supplementation in delaying onset of diabetes in female NOD mice observed up to 60 weeks post partum. Maternal diet treatments were either control or taurine supplemented in drinking water.

FIG. 16 illustrates islet histology from 14 day old mice with and without gestational taurine supplementation.

FIG. 17 illustrates apoptosis in islets from NOD female mice with and without gestational taurine supplementation.

FIG. 18 illustrates the percentage of islet cells testing positive for IGF-2 immunoreactivity from NOD female mice with and without gestational taurine supplementation.

FIG. 19 shows the insulin/glucagon ratio for small islets in NOD female mice with and without gestational taurine supplementation.

FIG. 20 shows the percentage of area stained for glucagon in small islets from NOD female mice with and without gestational taurine supplementation.

FIG. 21 shows the percentage of area stained for insulin in small islets from NOD female mice with and without gestational taurine supplementation.

FIG. 22 shows the percentage of PCNA positive cells in small islets from NOD female mice with and without gestational taurine supplementation.

FIG. 23 illustrates survival plots from NOD mice with and without gestational taurine supplementation.

### DETAILED DESCRIPTION OF THE INVENTION

The composition and method of the invention relate to inhibition of islet dysfunction. Through administration of a sulfur-containing amino acid to an individual in need thereof, conditions of islet dysfunction may be inhibited. The invention is now described in more detail, providing specific examples which are not to be considered limiting to the invention.

By “inhibiting islet dysfunction”, it is meant to ameliorate, treat, lessen, reverse, or prevent islet dysfunction, or to delay onset of islet dysfunction.

The term “islet dysfunction” refers to any condition which alters normal function, development or ontogeny, of islets or individual β cells. Exemplary conditions of islet dysfunction include but are not limited to insulitis, Type 1 diabetes, Type 2 diabetes, mature onset diabetes of the young (MODY), gestational diabetes, and developmentally-associated deficiencies in β cell number or function. Such developmentally-associated deficiencies in β cell number or function may result either from genetic abnormality or from environmental influences such as hypoxemia in utero, and prenatal or childhood nutritional deficiency or imbalance. Stages leading up to the development of any of the above-noted exemplary conditions of islet dysfunction are also considered within the realm of “islet dysfunction”.

By “inhibiting an autoimmune disorder”, it is meant to ameliorate, treat, lessen, reverse, or prevent an autoimmune disorder, or to delay onset of an autoimmune disorder.

The term “autoimmune disorder” refers to any condition which alters normal autoimmune function in a tissue-specific manner through lymphocyte and/or macrophage infiltration. Exemplary conditions of such autoimmune disorders which are within the scope of the invention include but are not limited to insulitis, type I diabetes, rheumatoid arthritis, thyroiditis, and pancreatitis. Such autoimmune disorders may result from or become exacerbated by environmental influences such as hypoxemia in utero, and prenatal or childhood nutritional deficiency or imbalance. A stage leading up to the development of any of the above-noted exemplary autoimmune disorders is also considered within the realm of an “autoimmune disorder”.

The term “biologically acceptable carrier” refers to any diluent, excipient, additive, or solvent which is either pharmaceutically accepted for use in the mammal for which a composition is formulated, or nutraceutically acceptable for use in a food product or non-drug dietary supplement. Further details of such carriers and dosage forms are provided below.

The term “amino acid like structure carrying a sulfur moiety” refers to those biologically acceptable compounds having adequate biological effect according to the invention, and includes sulfur-containing amino acids, sulfur derivatives of amino acids, derivatives of sulfur-containing amino acids, steriochemical isomers of sulfur-containing amino acids, tautomers of sulfur-containing amino acids, peptidomimetic derivatives of sulfur-containing amino acids, esters of sulfur-containing amino acids, and salts of any of the above compounds.

The term “sulfur-containing amino acid” refers to any biologically acceptable form of an amino acid containing a —SH, —S—, —SO₂⁻, or —SO₃⁻ moiety. The amino acid may be an α amino acid in levorotary form (L-α-), or may be a β amino acid. The acid moiety may be either CO₂⁻, as in the case of methionine, for example, or may be SO⁻₃, as in the case of taurine. The sulfur-containing amino acid may be in free base form, or may be delivered as a conjugate or peptide, as discussed in further detail below.

The invention is not limited to inhibition of islet dysfunction as effected through any particular mechanism of action. However, an exemplary mode of action through which islet dysfunction can be inhibited is through anti-apoptotic activity exerted by the amino acid like structure carrying a sulfur moiety, for example a sulfur-containing amino acid. A further exemplary mode of action through which islet dysfunction can be inhibited is through immunomodulatory activity exerted by the amino acid like structure carrying a sulfur moiety, for example by a sulfur-containing amino acid.

The invention is not limited to inhibition of autoimmune disorders through any particular mechanism of action. However, an exemplary mode of action through which autoimmune disorders can be inhibited is through anti-apoptotic activity exerted by the amino acid like structure carrying a sulfur moiety, for example a sulfur-containing amino acid. A further exemplary mode of action through which an autoimmune disorder can be inhibited is through immunomodulatory activity exerted by the amino acid like structure carrying a sulfur moiety, for example by a sulfur-containing amino acid.

By the term “anti-apoptotic activity” it is meant an activity resulting in prevention and/or delay of programmed cell death (apoptosis). To evaluate anti-apoptotic activity, any conventional measurement may be used, such as for example the TUNEL method as described below. A stimulator of cell death, such as for example sodium nitropruside (SNP) or IL1β, may be used to induce apoptosis in a model for evaluating anti-apoptotic activity. Prevention or delay of a naturally occurring apoptotic state, such as developmental β cell apoptosis, or an induced apoptotic state would be considered “anti-apoptotic activity”.

By the term “immunomodulatory activity” it is meant an activity resulting in alterations to an immune response. As it pertains to Type 1 diabetes, which is conventionally known to be an autoimmune disorder, “immunomodulatory activity” refers to alteration of such activities as: immune attack of β cells or islets; infiltration of lymphocytes or macrophages to β cells or islets (such as the condition known as insulitis); or a cytokine response from the immune system which exerts physiological effects on β cells or islets.

Amino Acid Like Structures Carrying a Sulfur Moiety. The amino acid like structures carrying a sulfur moiety according to the invention include sulfur-containing amino acids, as well as sulfur derivatives of amino acids, pharmacologically acceptable derivatives of sulfur-containing amino acids, steriochemical isomers of a sulfur-containing amino acids, tautomers of a sulfur-containing amino acids, peptidomimetic derivatives of a sulfur-containing amino acids, esters of amino acids, and salts thereof. Any derivative or analog of an amino acid incorporating a sulfur group, such as a designer amino acid, having an effect on inhibition of islet dysfunction or autoimmune disorders falls within the scope of the amino acid like structure carrying a sulfur moiety, according to the invention.

The sulfur-containing amino acid may be any physiologically acceptable form of an amino acid containing a —SH, —S—, —SO₂⁻ or —SO₃⁻ moiety. The sulfur-containing amino acid may be an a amino acid in levorotary form (L-α-), or may be a β amino acid. The acidic moiety may be either CO₂⁻, as in the case of methionine, for example, or may be SO₃⁻, as in the case of taurine, for example. The sulfur-containing amino acid may be in free base form, or may be delivered as a conjugate or peptide. In an exemplary embodiment, the sulfur-containing amino acid is selected from taurine, L-cysteine, L-methionine, and mixtures thereof.

According to the invention, the amino acid like structures carrying a sulfur moiety may be provided in a biologically acceptable conjugated form, for example a form which easily dissociates in aqueous solution. An exemplary conjugated for may be, for example, a hydrohalide form which may be either anhydrous, for example cysteine hydrochloride (HS—CH₂—CH(NH₃+)—CO₂⁻.HCl) or hydrated, for example cysteine hydrochloride monohydrate (HS—CH₂—CH(NH₃+)—CO₂⁻.HCl.H₂O). A conjugated sulfur-containing amino acid may be present in the composition as a pharmaceutically acceptable metal salt, such as for example a divalent metal taurate of the formula (H₂N—CH₂—CH₂—SO₃⁻)₂.X²+, where X²+ is magnesium or calcium.

Further, the amino acid like structures carrying a sulfur moiety may be delivered as a peptide having from two to five amino and acid residues bound together with peptide linkages. In such a peptide, the majority of the amino acids are sulfur-containing, and the dosage is calculated on the basis of the sulfur-containing amino acid residue content.

Dosage. The optimal dosage of an amino acid like structure carrying a sulfur moiety according to the invention, comprises a daily quantity of from about 0.5 grams and about 10 grams for a 50 kg human. A preferred daily dose is about 5 grams per day, or about 100 mg/kg, when expressed on a body weight basis. The dosage may be administered once daily, or throughout the day in fractions of the daily dose.

Dosage Forms. According to the invention, the composition comprises an amino acid like structure carrying a sulfur moiety and a biologically acceptable carrier. Examples of such carriers are provided below with reference to the diluents, excipients, solvents or additives relevant to a particular dosage form. The composition may be administered in a variety of dosage forms for either oral administration, parenteral infusion or injection. For oral administration, the composition may be provided as a tablet, an aqueous or oil suspension, a dispersible powder or granule, an emulsion, a hard or soft capsule, a syrup or an elixir. Compositions intended for oral use may be prepared according to any method known in the art for the manufacture of pharmaceutical or nutritional supplement compositions. One or more pharmaceutically acceptable excipients suitable for tablet manufacture may be added to the composition. Exemplary excipients include inert diluents such as calcium carbonate, sodium carbonate, lactose, calcium phosphate or sodium phosphate; granulating and disintegrating agents, such as corn starch or alginic acid; binding agents such as starch, gelatin or acacia; and lubricating agents such as magnesium stearate, stearic acid or talc. The composition of the invention may contain one or more additive, such as a sweetener, a flavoring agent, a coloring agent or a preservative to increase the palatability or consumer appeal of the composition. Such a composition may contain a preservative, such as an antioxidant, for example ascorbic acid. Tablets may be coated or uncoated, and may be formulated to delay disintegration in the gastrointestinal tract and thereby provide sustained release. For example, a time delay material such as glyceryl monostearate or glyceryl distearate alone or with a wax may be incorporated into the composition.

Oral dosage forms of the composition may also be provided as a gelatin capsule wherein the amino acid like structure carrying a sulfur moiety, for example a sulfur-containing amino acid, is mixed with an inert solid diluent, for example calcium carbonate, calcium phosphate or kaolin, or with water or an oil medium, such as peanut oil, liquid paraffin or olive oil. Aqueous suspensions may contain the amino acid like structure carrying a sulfur moiety in admixture with one or more excipient suitable for the manufacture of an aqueous suspension, for example a suspending agent, a dispersing or wetting agent, a preservative, a coloring agent, a flavoring agent or a sweetening agent such as sucrose, saccharin or aspartame. An oil suspension may be formulated by suspending the amino acid like structure carrying a sulfur moiety in a vegetable oil, such as olive oil, sesame oil or coconut oil, or in a mineral oil such as liquid paraffin. The oil suspension may contain a thickening agent, such as cetyl alcohol. A sweetening, flavoring, or coloring agent may be added to increase palatability or consumer appeal. Such a composition may contain a preservative, such as an antioxidant, for example ascorbic acid.

Dispersible powders and granules of the invention suitable for preparation of a suspension by the addition of an aqueous solute provide an amino acid like structure carrying a sulfur moiety in combination with a dispersing or wetting agent, a suspending agent, and one or more preservatives. Suitable aqueous solutes include water, milk, fruit juice, etc. Additional excipients, for example sweetening, flavoring and coloring agents, may also be present. These dispersible powders further increase the appeal to the consumer, as they can be incorporated into a selected liquid component of an individual's routine diet, in the case where an individual is adverse to swallowing a pill or a capsule.

The composition may be formulated as a syrup or elixir according to methodologies known in the art to combine sulfur-containing amino acids with sweetening agents, such as glycerol, sorbitol or sucrose. Such formulations may also contain a preservative, a flavoring or a coloring agent.

Sulfur-containing amino acid preparations for parenteral administration may be in the form of a sterile injectable preparation, such as a sterile injectable aqueous suspension or emulsion. Such preparations are formulated according to methodologies known in the art using suitable dispersing agents, wetting agents, suspending agents, diluents or solvents. Suitable diluents or solvents include water, Ringer's solution and isotonic sodium chloride solution. In addition, sterile fixed oils may be employed conventionally as a solvent or suspending medium. For this purpose, any bland fixed oil may be employed including a synthetic monoglyceride or diglyceride. In addition, fatty acids such as oleic acid may likewise be used in formulating injectable preparations.

The dosage form may also be an oil-in-water emulsion. The oil phase may be a vegetable oil, such as olive oil, a mineral oil such as liquid paraffin, or a mixture thereof. Suitable emulsifying agents include naturally-occurring gums such as gum acacia or gum tragacanth; naturally occurring phosphatides, such as soybean lecithin; esters or partial esters derived from fatty acids and hexitol anhydrides, such as sorbitan mono-oleate; and condensation products of these partial esters with ethylene oxide, such as polyoxyethylene sorbitan mono-oleate. An emulsion may also contain sweetening and flavoring agents.

When prepared as a pharmaceutical preparation, the invention includes such formulations which combine an amino acid like structure carrying a sulfur moiety, for example a sulfur-containing amino acid, with other pharmaceutically active ingredients, such as other drugs targeting the diabetic or pre-diabetic condition.

The composition according to the invention may also be prepared as a nutritional-supplement in combination with any ingredient such as vitamins, minerals, amino acids, dietary fiber or other dietary component which would be considered biologically acceptable. Food-grade ingredients which are generally recognized as safe may be included in the composition. Such a nutritional supplement could be provided in a tablet form as well as in a food form, such as in a shake, a bar, or in a powder intended for hydration in a food-grade liquid such as milk or fruit juice. Further, the composition may be added directly to food, such as into a fruit juice or milk product, in which case the carrier (ie—the food) would be considered biologically acceptable from a pharmaceutical and nutraceutical perspective.

The inventive composition may be formulated as a pharmaceutical or nutraceutical product or supplement, or as a functional food or infant formula, in combination with other active ingredients to produce an additive or synergistic effect. For example, a composition containing an amino acid like structure carrying a sulfur moiety, for example a sulfur-containing amino acid, could also contain nicotinamide. Such a combination could be used, for example, to inhibit islet cell death in type I diabetes.

The inventive composition may be formulated as an infant formula to be given to an infant as a complete nutritional product. Such infant formulae are known in the art. Such an infant formula composition could also contain other active ingredients, such as nicotinamide as noted above, in sufficient amounts to provide an additive or synergistic effect on inhibition of islet dysfunction or inhibition of an autoimmune disorder.

The invention is intended for use by mammals susceptible to islet dysfunction or autoimmune disorders. The mammal may be a human, but may also be a laboratory, agricultural or domestic mammal which may benefit from the invention. The invention may be implemented for human individuals who have developed or who are at risk to develop conditions of islet dysfunction or autoimmune disorders, or whose offspring may be susceptible to development of such conditions or disorders. The invention may be thus used as a maternal treatment or supplement, as a supplement to lactating mothers, as a component of an infant formula, or may be delivered directly to infants or children during youth, or later in life when risk of Type 2 diabetes is increased.

Delivery of synthetic enzymes or gene therapies which are capable of altering in vivo conversion or metabolism of sulfur-containing amino acids such as taurine and cysteine so as to have a net effect of increasing or altering sulfur-containing amino acid content within one or more tissues of the body would also be considered within the realm of the invention.

### EXAMPLES

The following examples are to be viewed as instructive and illustrative of the invention, and are in no way limiting. All experimental results reported herein are means±SEM, unless otherwise indicated. Significance of difference between groups was analysed by Scheffe's Test after a one-way or two-way ANOVA. The L-form of the α-amino acids was used throughout all methodologies described herein.

### Example 1

### Apoptosis in Cultured Fetal Islets

Animals and Diets. Adult virgin female Wistar rats were caged overnight with males and copulation was verified the next morning. Animals were maintained at 25° C. with a 10 h-14 h dark-light cycle. Pregnant rats were divided into two groups and fed one of the following isocaloric diets: either a control diet (C) containing 20% protein or a low protein diet (LP) containing 8% protein. The composition of the diet was as described previously by Snoeck et al. 1990 (Biol. Neonate 57, 107-118). The diets were purchased from Hope Farms (Woerden, Holland). Animals in both the groups had free access to water at all times. At 21.5 days of gestation, females were sacrificed by decapitation and fetuses removed.

Islet culture and treatment. Fetal islets were isolated as described by Mourmeaux et al. 1985 (Molecular and Cellular Endocrinology 39:237-246). Islets were cultured in 35 mm Petri-dishes (Falcon plastics, Los Angeles, Calif.) in RPMI 1640 medium (Gibco, Grand Island, N.Y., USA) with 10% Fetal Bovine Serum and antibiotics (penicillin 200 U/ml, streptomycin 0.2 mg/ml). Petri dishes were incubated at 37° C. with 5% CO₂ in air. The culture medium was changed daily after the second day. On the 5th day of culture, islets were rinsed twice with serum free DME/F12 medium (1:1, v/v, Gibco, Paisley, Scotland) and subsequently incubated for 42 hours in this medium. After seven days of culture, neoformed islets mainly comprised β cells (>95%).

Sodium nitropruside (SNP) treatment of cultured islets. A pathway which has been proposed as being the effector for IL1β-induced apoptosis is the stimulation of inducible NO synthase. The proinflammatory cytokines TNα A and IFNγ also induce NO formation in β cells and other interacting islet cell types, such as macrophages, endothelial cells and fibroblasts. These cytokines likely synergize to maximize β cell destruction. The ability of IL 1 β, TNF-α and IFN-γ to induce NO synthesis causing β cell death is mediated by apoptosis. Thus, to examine the effect of NO on apoptosis in islet cells, SNP, a NO donor, was added to cultured islets. Islets were cultured islets in RPMI 1640 medium with or without SNP at levels of 0, 10, and 100 μmol/l for 18 hours.

TUNEL method for evaluating apoptosis. Apoptosis was evaluated by the TUNEL method described herein, and visualized with confocal microscopy. Cultured islets were fixed in methanol and stored at −20° C. until analysis. The tissue was then washed with phosphate buffered saline (PBS) for 3 min, and a terminal deoxynucleotidyl transferase (TdT) reaction buffer (50 μl) was added. The 50 μl of TdT solution was prepared using 10 μl of 5× concentrated buffer solution (1 mol/l potassium cacodylate; 125 mmol/l Tris-HCl, pH 6.6; 1.26 mg bovine serum albumin). Cobalt chloride (5 μl of 25 mmol/l), 0.5 μl (12.5 units) of TdT (both from Boehringer Mannheim, Germany), and 0.25 nmoles of BODIPY-FL-X-14-dUTP (Molecular Probes, Eugene, Oreg. USA) were added, along with distilled water, up to 50 μl. Islets were incubated in Petri dishes with the TdT reaction buffer for 60 min at 37° C., then rinsed twice with 15 mmol/l EDTA (pH 8.0) in PBS and once with 0.1% Triton X-100 in PBS. Then, 2 ml of PBS containing 2.5 μg/ml of propidium iodide (Molecular Probes, Eugene, Oreg. USA) was added to the dishes for 5 min. Finally, islet cells were washed with 15 mmol/l EDTA (pH 8.0). Islet cells were then double-labelled for apoptotic nuclei, showing the BODIPY-FL-dUTP label in yellow and total nuclei with propidium iodide in red.

Confocal microscopy. Staining probes were visualised through a confocal laser scanning microscopy system (MRC-1024 UV; BIO-RAD, UK) equipped with Argon ion and Krypton/Argon ion lasers. BODIPY-FL was excited at 503 nm, ethidium bromide at 510 nm, propidium iodide at 536 nm and Hoechst 33342 at 346 nm. The emissions were recorded respectively at 522/32 nm, 605/32 nm and 455/30 nm. Four to six optical sections were collected at every 15 μm through the islet. The number of BODIPY-FL-positive or ethidium bromide-positive nuclei were reported and expressed as a percentage of the total number of nuclei.

Global cell death. Global cell death was analysed using a non-specific staining permeant probe. For this purpose, the culture medium was removed and the dishes were incubated in the dark with 1 ml of 20 μg/ml ethidium bromide for 20 min to stain permeabilized dead cells. The cultures were then fixed with 4% paraformaldehyde in PBS for 10 min, treated with 30% methanol for permeabilization of the remaining of the cells and then mounted in mowiol containing 20 μg/ml of Hoechst 33342, to stain the nuclei.

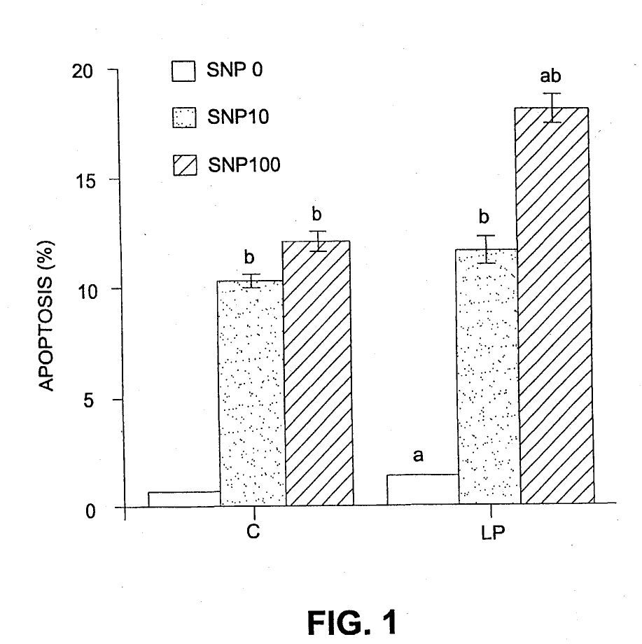

FIG. 1 shows that, in the absence of SNP (SNP 0 group), islet cell apoptosis was significantly higher in the low protein group (LP) compared with the control (C) group. Thus, a low protein diet during gestation increased the susceptibility of fetal islets to apoptosis even in the absence of inducement from an NO donor. Values are the means of at least 28 islets pooled from 3 different cultures with at least 2000 cells/group. The letters positioned above the bars indicate statistical significance as follows: a: p<0.01 C vs LP; b: p<0.01 SNP 0 vs both SNP 10 and SNP 100.

Further, SNP-induced islet cell apoptosis was significantly higher in the low protein diet group (LP) than in the control (C) group. The rate of islet apoptosis increased in a dose-dependent manner between the 10 μmol/l and the 100 μmol/l SNP treatments, for both diet groups. This effect was more severe in LP islets at the high SNP concentration, and it can be seen that at 100 μmol/l SNP, apoptosis was significantly higher in islet cells from the LP group than from the C group. To confirm this result, the percentage of mortality in response to 100 μmol/l SNP was measured using a test for cell permeability to ethidium bromide. LP fetal islets showed 10.4±0.7% mortality while control islets featured only 2.9±0.8%, which corroborates the result obtained using the TUNEL method.

SNP is a complex of ferrous iron (Fe²⁺) with five cyanide anions (CN⁻) and a nitrosonium (NO⁺) ion. SNP can simultaneously liberate nitric oxide and an iron moiety capable of generating .OH radicals. In order to verify that cytotoxicity of SNP was not mainly due to this reactive species, desferioxamine (DFO), an iron chelator was tested. DFO partially reduced the apoptotic rate for both LP and C islets, but this reduction accounted only for about 30% of the cell death induced by SNP at the 100 μmol/l level (data not shown). The major part of the toxic effect of SNP is thus attributable to NO, to which LP islets are more sensitive than C islets. This result shows that protein deprivation during gestation increases the sensitivity of the β cell mass to nitric oxide.

### Example 2

### Taurine Content of Cultured Fetal Islets

Animals, diets, and fetal islet isolation procedures were conducted as described in Example 1. The concentration of taurine after seven days of culture was measured in islets by the following HPLC method. Islets were incubated overnight at +4° C. in 35% 5-sulfosalicylic acid in order to extract amino acids therefrom. Separation and quantification of the amino acids was performed with a standard, reverse phase HPLC method after derivatization with o-phthalaldehyde. Low protein (LP) islets showed a significantly lower taurine concentration of 22.9±1.55 mmol/μg of protein as compared with control islets having a concentration of 36.9±5.22 mmol/μg of protein (p<0.01).

### Example 3

### Effect of Taurine, Methionine and β-Alanine on Apoptosis in Fetal Islets

Animals, diets, and fetal islet isolation procedures were conducted as described in Example 1. On the 5th day of culture, islets from animals fed a control diet (C) or a low protein diet (LP) were rinsed twice with serum free DME/F12 medium (1:1, v/v, Gibco, Paisley, Scotland) and incubated for 48 hours in this medium supplemented with or without an amino acid selected from taurine, methionine and β-alanine. To determine if the activity of taurine was specific to its particular amino acid structure, the effect of methionine and β-alanine on the islet cells apoptosis induced by SNP was examined for comparison. All supplemented amino acids were purchased from Sigma Chemical Co. (St Louis, Mo.).

Physiological and supraphysiological levels of each amino acid were tested. Taurine, if present was either 0.3 mmol/l (physiological) or 3 mmol/l (supraphysiological); methionine, if present was either 0.1 mmol/l (physiological) or 1 mmol/l (supraphysiological); and β-alanine, if present was either 0.3 mmol/l (physiological) or 3 mmol/l (supraphysiological). Supplemental amino acid levels were maintained in the culture medium during SNP treatment. In the final 24 hours of the 48 hour incubation, SNP (100 μmol/l) was added. Apoptosis was quantified by confocal microscopy using the TUNEL method described in Example 1. Mortality rate was quantified by confocal microscopy using permeant probes, as described in Example 1, to verify quantification of apoptotic rate for both C and LP diet treatments including SNP and taurine.

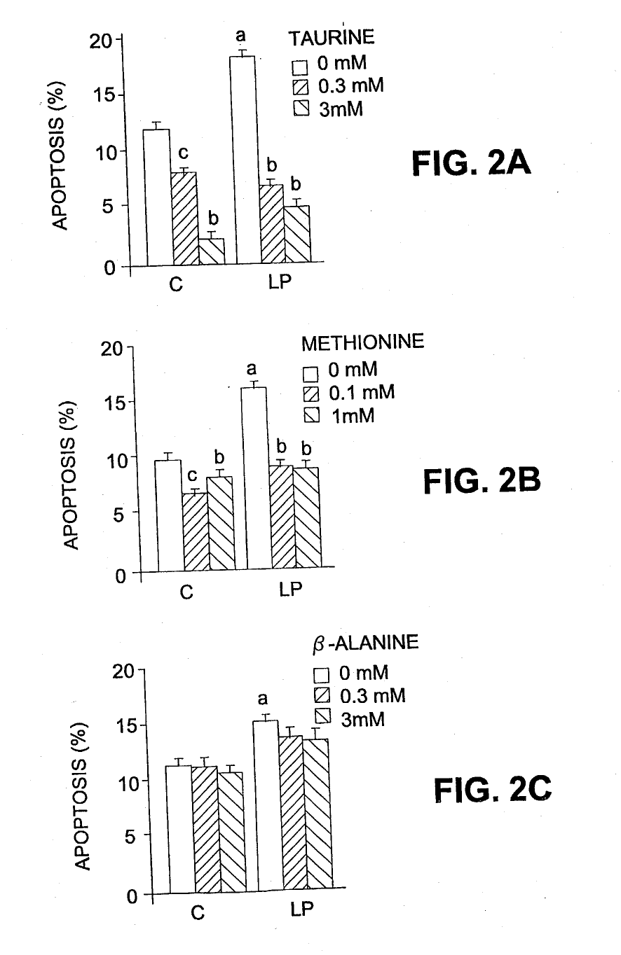

FIG. 2 illustrates that taurine is protective against SNP-induced apoptosis in vitro. Values are the means of at least 28 islets pooled from 3 different cultures with at least 3500 cells/group. The letters above the bars indicate statistically significant differences as follows: a: p<0.01 C vs LP; b: p<0.01 for 0 mmol/l taurine vs 0.3 or 3 mmol/l taurine, and p<0.01 for 0 mmol/l methionine vs 0.3 or 3 mmol/l of methionine; and c: p<0.05 for 0 mmol/l vs 0.3 mmol/l. At physiological or supraphysiological concentration, taurine significantly decreased the percentage of β cells positive for apoptosis in both groups. However the protective effect of a physiological concentration (0.3 mmol/l) of taurine was more marked in the LP islets (60% reduction of apoptosis vs 30% in controls).

Further, the apoptosis rate was significantly decreased when methionine was used at physiological concentration (0.1 mmol/l methionine) regardless of diet. At a supraphysiological concentration (1.0 mmol/l) methionine did not provide additional protection, beyond that of the physiological concentration. Thus, it is clear that methionine also exerts a protective effect in fetal β cells against the cytotoxicity induced by SNP as a NO donor, although this effect was less marked than that of taurine. The rate of apoptosis was similar with or without β-alanine, indicating that β-alanine exerted no protective effect on the fetal β cell against damage induced by NO.

By way of comparison with the data of FIG. 2, in the absence of SNP, taurine-treated islets from animals fed a control diet (C) exhibited in vitro apoptotic rates of 1.5±0.2% (0.3 mmol/l taurine) and 1.4±0.3% (3 mmol/l taurine), which were not significantly different from the islets incubated without taurine, having a rate of 1.3±0.2% (0 mmol/l taurine). Under taurine-free in vitro conditions, islets isolated from animals fed a low protein diet (LP) demonstrated an apoptotic rate approximately two-fold higher (2.2±0.3%) than that of islets isolated from animals fed a control diet (C). As with the islets from animals fed a control diet, the presence of taurine in the incubation medium did not affect the apoptotic rate in islets isolated from animals fed a low protein diet (2.1±0.3% and 2.1±0.4%, with 0.3 and 3 mmol/l of taurine, respectively for LP).

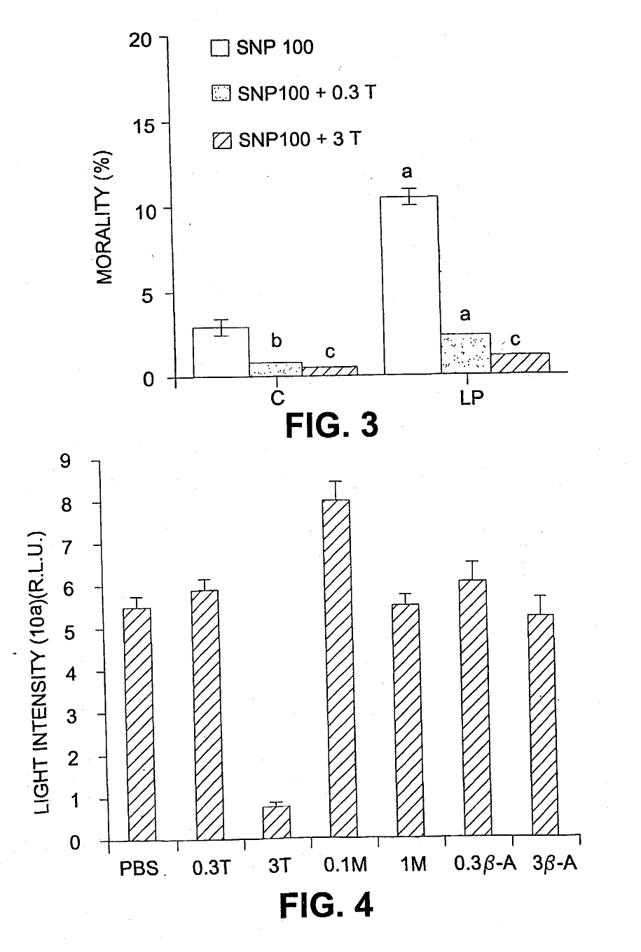

FIG. 3 shows that the mortality after treatment with SNP (100 mmol/l), expressed as percentage of cell death, is significantly diminished when islet cells are pre-treated with taurine at either physiological or supraphysiological concentrations. This was true for both diet groups, and the effect is dose dependent. The letters above the bars indicate statistically significant differences as follows: a: p<0.01 C vs LP; b: p<0.05 for 0 mmol/l taurine vs. 0.3 mmol/l taurine; c: p<0.010 mmol/l taurine vs 0.3 or 3 mmol/l taurine.

### Example 4

### NO Formation and Quenching of Peroxynitrite Formation in vitro

Nitrite assay. The concentration of NO was quantified in an acellular system in the presence of SNP alone or with taurine. Nitrite, a stable end product of NO oxidation, was measured by a fluorometric procedure, based upon the reaction of nitrite with the 2,3-diaminonaphtalene (DAN) (Molecular Probes) to form the fluorescent product 1-(H)-naphthotriazole. This method allows measurement of nitrite at levels as low as 10 nmol/l. In order to measure total NO production in the culture media, nitrate was converted to nitrite by the action of nitrate reductase from Aspergillus species (Sigma Chemical Co.). The sample (100 μl) was incubated with 100 μl of 20 mmol/l Tris buffer (pH 7.6) containing in final concentration 80 μmol/l NADPH (to initiate the reaction) and 56 mU of enzyme. The reaction was stopped after 5 min at room temperature by dilution with 1800 μl ultrapure water, followed by the addition of the DAN reagent (200 μl of a 0.05 mg/ml solution in 0.62 mol/l HCl). Finally, 100 μl of 2.8 mol/l NaOH was added to each sample. Nitrite concentration was determined using sodium nitrite (Sigma Chemical Co.) as a standard. The fluorescence was measured in a Kontron fluorimeter at excitation and emission wavelengths of 365 nm and 450 nm, respectively.

Chemiluminescence measurements. Luminol (5-amino-2,3-dihydro-1-4-phtalazinedione) at 400 μmol/l (Sigma Chemical Co.), taurine (0.3 or 3 mmol/l), methionine (0.1 or 1 mmol/l) and β-alanine (0.3 or 3 mmol/l) stock solutions were prepared in PBS. Sydnonimine (SIN-1), also known as 3-morpholinosydnonimine, a source of peroxynitrite, was purchased from Sigma Chemical Co. SIN-1 was prepared as 100 μmol in 1 mol/l NaOH. A reaction was initiated by simultaneous injection of luminol and SIN-1 into wells containing PBS either alone or with supplemental amino acid levels as noted above. Chemiluminescence emitted by luminol in the presence of peroxynitrite was measured every 30 seconds over 20 minutes in a chemifluorophotometer (MicroLumat LB96P, EG&G BERTHOLD), and was quantified as light intensity (10⁸ RLU).

Nitric oxide is a reactive free radical which leads to peroxynitrite formation, another reactive free radical, by interaction with superoxide (NO+OO^{-→}ONOO⁻). To determine the mode of action through which taurine exerts a protective effect, the possible direct molecular interaction between taurine and the NO donor, and the formation of peroxynitrite were investigated. The concentration of NO in an acellular system was quantified in the presence of SNP alone or with taurine. The concentration of NO released in the presence of SNP was not significantly altered by taurine in vitro at 0.3 mmol/l or 3 mmol/l. Luminol-derived chemiluminescence induced by peroxynitrite produced by the decomposition of sydnonimine (SIN-1) was evaluated to investigate the possibility that taurine quenched the peroxynitrite formed from NO.

FIG. 4 shows the effect of addition of taurine (0.3 or 3 mmol/l), methionine (0.1 or 1 mmol/l) or β-alanine (0.3 or 3 mmol/l) in this system, providing the means±SEM of seven replicates. At 3 mmol/l of taurine, luminol chemiluminescence, representing peroxynitrite quenching, was dramatically decreased. No change was observed when methionine was added. Further, in vitro additions of β-alanine showed no change in chemiluminescence.

### Example 5

### Effect of Taurine on IL1β-Induced Apoptosis

IL1β alone or in combination with TNFα plus IFNγ induces apoptosis in β cells. IL 1β is a predominant macrophage-derived proinflammatory cytokine. Exposure of rat islets in vitro to exogenous IL 1β induces a transient increase in glucose-stimulated insulin release, although prolonged in vitro exposure decreases β cell insulin synthesis, reduces the DNA synthetic rate of fetal or neonatal islets, and results ultimately in cell death. Administration of high doses of IL 1β accelerates Type 1 diabetes while low doses prevent Type 1 diabetes in BB rats (Wilson et al. J. Immunol. 1990;144: 3784). Treatment of NOD mice with soluble IL1β significantly delays Type 1 diabetes onset (Nicoletti et al. Eur. J. Immunol. 1994; 24:1843). The ability of IL1βto initiate β cell damage is believed to be dependent on signalling, mRNA transcription, de novo protein synthesis and diminished mitochondrial function.

The effect of in vitro taurine on the effect of IL1β-induced apoptosis was assessed. Fetal islets were isolated as described above in Example 1. On the 5th day of culture, islets were rinsed twice with serum free DME/F12 medium (1:1, v/v, Gibco, Paisley, Scotland) and were incubated in this medium supplemented with taurine at 0.3 or 3 mmol/l for 48 hours. For the final 24 hours of this incubation, IL1β (Endogen, Woburn, Mass.) was added to the incubation medium at a level of 50 U/ml. Apoptosis was quantified by confocal microscopy using TUNEL method, as outlined in Example 1.

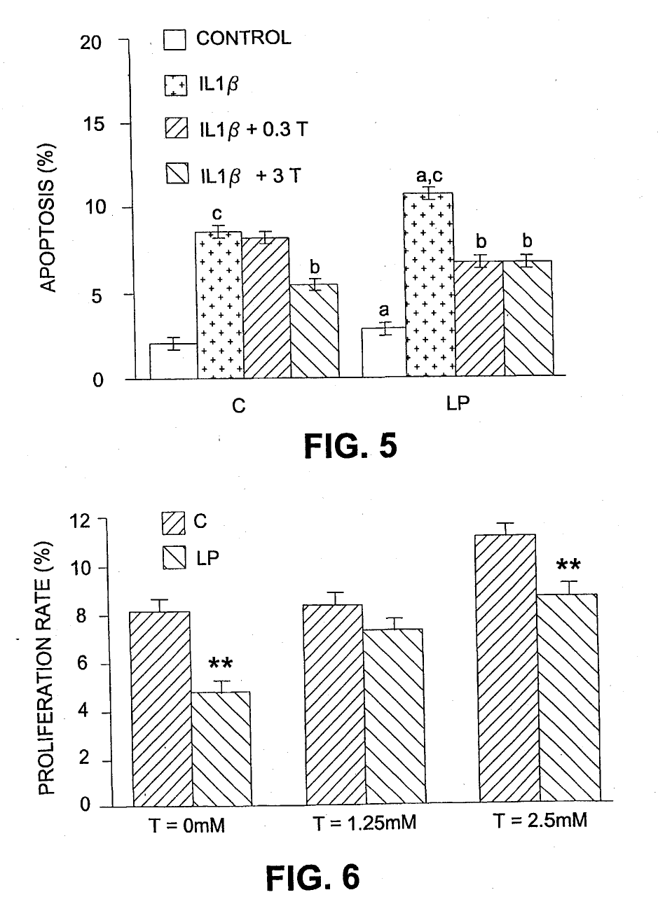

FIG. 5 provides the results of this experiment, illustrating % apoptosis in the presence of taurine. Values are the means of at least 28 islets pooled from 3 different cultures with at least 2000 cell/group. In this experiment, the basal rate of apoptosis in both groups was somewhat higher than was illustrated in Example 4. Incubation of fetal islets with 50 U/ml IL 1β for 24 hours increased the apoptosis level in the C group and even more than in the LP group. For the C diet group, only the high dose of taurine decreased significantly the rate of apoptosis in islet cells. In the LP diet group, taurine at physiological (0.3 mmol/l) and supraphysiological (3.0 mmol/l) concentrations significantly decreased the number of islet cells positive for apoptosis. The letters above the bars illustrate significant differences as follows: a: p<0.05 C vs LP; b: p<0.0 IL1β alone vs addition of taurine; c: p<0.01 control (no IL 1β) vs IL1β. This illustrates that a low protein diet during gestation increases the susceptibility of fetal β cells to cytokine IL 1β, and that in vitro incubation with taurine reduced the susceptibility imposed on islets of offspring by maternal dietary treatment. This example illustrates that although increased apoptosis following IL1β exposure was observed in both diet groups (C and LP), the increase in apoptosis for the LP islet cells was higher than in C islet cells. It is clear that low protein diet during gestation augments the sensitivity of fetal β cells to IL 1β, and that in vitro taurine can ameliorate this increased sensitivity.

### Example 6

### Effect of in vitro Taurine on Islet Cell Proliferation

Animals and diets were as described in Example 1. Fetuses were removed at fetal day 21.5, and fetal islet cells were isolated and treated as described in Example 1. Taurine was added to incubation medium at levels of either 0 mmol/l, 1.25 mmol/l or 2.5 mmol/l. Proliferation was evaluated using bromodeoxyuridine (BrdU) incorporation into DNA.

FIG. 6 shows that in islet cells from rats consuming a low protein diet, in vitro proliferation rate was suppressed compared to control animals. Taurine additions to culture media in vitro increased LP islet cell proliferation rate to the levels observed in C islets having no taurine addition. This observation demonstrates that in vitro taurine can counteract the reduction in proliferation rate induced by feeding a low protein diet.

### Example 7

### Islet Cell Proliferation With Dietary Taurine Supplementation

Adult virgin female Wistar rats were caged overnight with males and copulation was verified the next morning. Animals were maintained at 25° C. with a 10 h-14 h dark-light cycle. Pregnant rats were divided into four groups and fed one of the following isocaloric diets, either with or without taurine supplemented in the drinking water. The control group (C) consumed a basal control diet containing 20% protein, the control plus taurine supplemented group (C+Taurine) consumed a basal control diet containing 20% protein, supplemented with taurine in the drinking water at a level of 2.5% (weight/volume), the low protein group (LP) consumed a low protein diet containing 8% protein, and the low protein plus taurine group (LP+Taurine) consumed an 8% protein diet supplemented with taurine in the drinking water at a level of 2.5% (weight/volume). The composition of the basal and low protein diets were described previously by Snoeck et a/.1990 (Biol. Neonate 57, 107-118). The diets were purchased from Hope Farms (Woerden, Holland). Animals in both the groups had free access to water at all times.

Animals were divided into four groups to assess various parameters at different time periods. Females were sacrificed at 21.5 days of gestation by decapitation and fetuses were removed in order to evaluate parameters at fetal day 21.5 (F 21.5). Alternatively, animals gave birth and offspring were sacrificed at post-natal day 12, 14 or 30 (PN 12, PN 14, and PN 30, respectively). Bromodeoxyuridine (BrdU) incorporation was evaluated by immunostaining as described by Petrik et al., (Endocrinology 1999;140: 4861-4873).

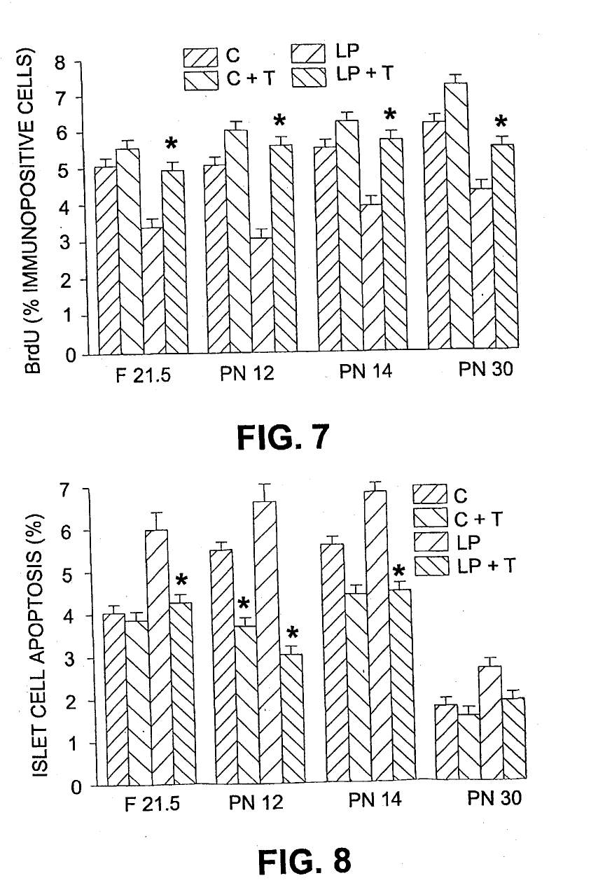

FIG. 7 shows that BrdU incorporation in islets from animals exposed to different maternal diets varied as a function of dietary taurine supplementation. Statistically significant differences between the taurine-supplemented and the non-taurine supplemented diet group within a treatment is indicated by (*) appearing above a bar. The data illustrate that a maternal LP diet reduces proliferation (as determined using BrdU incorporation) when compared to the C diet, but that taurine supplementation of a low protein diet (LP+Taurine) was able to restore the proliferation rate to a level not significantly different from the control diet. This illustrates the restorative property of supplemental taurine on proliferation of β cells in animals facing nutritional restriction. However, when no nutritional challenge is induced, such as for those animals consuming the control maternal diet, taurine supplementation did not significantly increase β cell proliferation rate. This indicates that sulfur-containing amino acids, and taurine in particular, are particularly effective in restoring a normal proliferative rate in β cells for animals challenged by nutritional deficiency.

### Example 8

### Dietary Taurine Reduces Islet Cell Apoptosis in Protein-Deprived Animals

Animals and diets were prepared, and taurine supplementation in drinking water was conducted as described in Example 7. Islet cells were cultured and treated as described in Example 1. Islet cell apoptosis was determined using the TUNEL method, as described by Petrik et al., (Endocrinology 1999;140: 4861-4873). Four developmental stages were evaluated, namely: fetal day 21.5 (F21.5) or post-natal day 12 (PN12), 14 (PN14) or 30 (PN30).

FIG. 8 illustrates that at each developmental stage the LP diet group exhibited increased apoptosis relative to the C diet group. Statistically significant differences between the taurine-supplemented and the non-taurine supplemented diet group within a treatment is indicated by (*) appearing above a bar. The increase in apoptosis due to protein level in the diet was ameliorated by the addition of taurine to the maternal diet through drinking water supplementation, and in each case, the taurine-supplemented low protein group (LP+Taurine) showed either no difference, or a reduction in apoptosis compared to the control group (C).

At post-natal days 12 and 14, which represent the time period at which the apoptosis rate of β cells increases naturally due to developmental apoptosis, the effect of taurine was particularly striking, as even the protein-sufficient (C) animals experienced a reduction in apoptosis when supplemented with taurine.

### Example 9

### Islet Cell Immunoreactivity With Dietary Taurine Supplementation

Insulin-like growth factors (IGFs) stimulate cell proliferation and differentiation in vitro, and control fetal size at birth. In the mid-trimester human fetus, levels of IGF-II mRNA in β cells are as much as 100-fold greater than levels of IGF-I. Isolated islets from the human and rat fetus or neonate express and release immunoreactive IGF-I and -II. There is much evidence that IGFs potentiate β cell growth, maturation, and function, and are expressed by β cells in early life. IGF-II mRNA is greatest in the fetal pancreas, being expressed within islet cells and focal clusters of ductal epithelial cells, but this level declines during the neonatal period.

The transient β cell apoptosis seen in the neonatal rat two weeks after birth coincides temporally with a diminished pancreatic expression of islet IGF-II (Petrik et al., Endocrinology 1998; 139: 2994-3004). IGF-I and -II are able to prevent apoptosis in a variety of cell types. Endogenous IGF-II within isolated neonatal rat islets is protective against cytokine-induced apoptosis. This protection is lost at weaning when islets no longer express IGF-II, but can be restored with exogenous IGF-II. Changes in IGF-II availability provoke developmental β cell apoptosis. While IGF-II has a role in the homeostasis of β cell mass in early life, it is predominantly a growth and survival factor for endocrine cells already formed.

Animals and diets were prepared as described in Example 7. The pancreas was removed, and IGF-II immunoreactivity was evaluated according to a method described by Petrik et al., (Endocrinology 1999;140: 4861-4873). Expression of IGF-II, considered a survival factor for cells, was measured as an indicator of the overall health of the β cells.

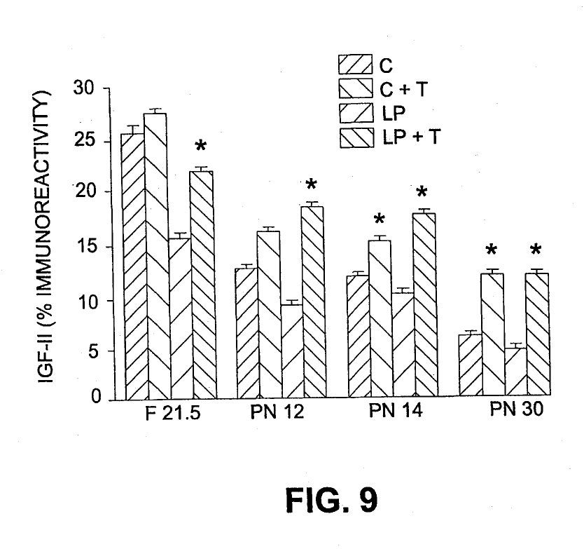

FIG. 9 illustrates that IGF-II expression was reduced by protein restriction, most markedly at fetal day 21.5. Statistically significant differences between the taurine-supplemented and the non-taurine supplemented diet group within a treatment is indicated by (*) appearing above a bar. Post-natal effects of protein restriction on IGF-II were less marked than fetal effects, probably because the IGF levels decrease in the post-natal animal. Supplementation of taurine in the fetal period increased IGF-II immunoreactivity to a level consistent with the C diet animals without taurine supplementation. Thus, taurine supplementation mitigated the negative effect of the LP diet on IGF-II at the fetal stage. Post-natal dietary supplementation of taurine for animals fed a low protein diet also restored IGF-II immunoreactivity levels to the point that the control IGF-II level was surpassed. These data further illustrate that the portion of islet cells demonstrating immunoreactive IGF-II in early life was decreased following exposure to a maternal LP diet, but was restored by maternal taurine supplementation, which appeared to delay the age-related loss of IGF-II in neonatal islets.

### Example 10

### Taurine Supplementation Effects on Fast Fas Ligand, iNOS and VEGF

The protective action of taurine on islet cells is less apparent when cells are treated with IL1β (as in Example 5) than with the NO donor (as in Example 3). This disparity may be attributable to stimulation of inducible isomers of the NO synthase enzyme (iNOS), leading to production of NO which mediates the cytotoxicity of IL1β towards β cells. Immunomodulatory activity can be evaluated using these parameters. IL 1β-induced loss of β cell function and viability are linked to NO production, and particularly to cytotoxic effects on mitochondrial function and DNA fragmentation. Specific inhibitors of NOS activity prevent IL 1β-induced changes in insulin release and β cell viability. Intra-islet release of IL1β following passenger macrophage activation promotes iNOS activity in β cells, and consequent damage.

It is known that IL1β can stimulate in vivo apoptosis of β cells by inducing Fas expression. When human islet cells are primed to undergo apoptosis by IL1β, it has been suggested that this involves the close association between cell-surface Fas and its ligand (Fas ligand). Further, it is hypothesized that IL1β in combination with TNFα and IFNγ induce β cells apoptosis by two independent pathways, namely NO production and Fas surface expression. Fas is a transmembrane cell surface receptor protein related to the TNFα receptor family. Activation by the Fas ligand results in an intracellular signaling cascade terminating in apoptosis. Thus, the effect of taurine supplementation on immunoreactivity of Fas and Fas ligand in the pancreas was assessed.

Vascular endothelial growth factor (VEGF) is involved in β cell ontogeny. VEGF is a potent mitogen for endothelial cells both in vitro and in vivo, and also increases vascular permeability. The effect of taurine supplementation on pancreatic VEGF immunoreactivity was assessed.

Animals and diets were prepared as described in Example 8. The pancreas was removed, and the presence of Fas, Fas ligand, inducible nitric oxide synthase (iNOS) and vascular endothelial growth factor (VEGF) were evaluated in pancreatic sections using immunoreactivity.

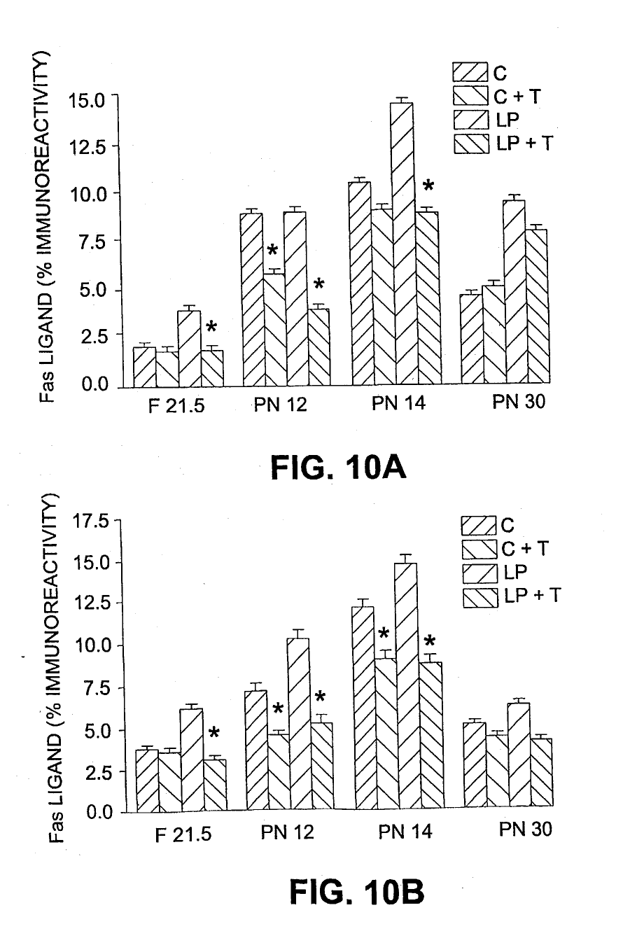

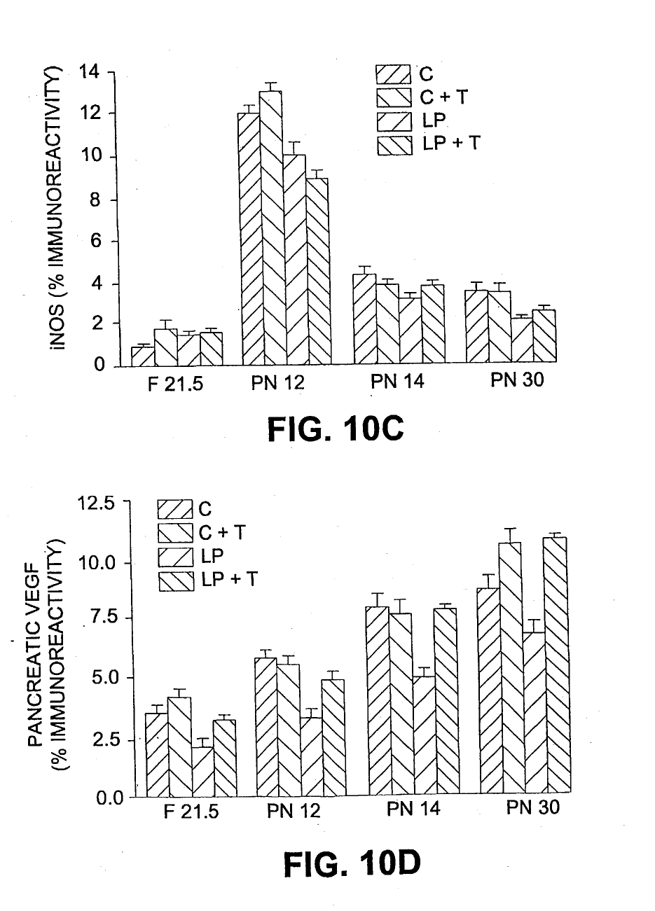

FIG. 10A to FIG. 10D illustrates the effect of a low protein diet, taurine supplementation and developmental stages on Fas, Fas ligand, iNOS and VEGF, respectively. Statistically significant differences between the taurine-supplemented and the non-taurine supplemented diet group within a treatment is indicated by (*) appearing above a bar. FIG. 10A indicates that a low protein diet causes an increase in the presence of immunoreactive Fas within islets. FIG. 10B further illustrates that a low protein diet causes an increase in Fas ligand. The greatest effect of the low protein diet was at the time of neonatal developmental apoptosis at post-natal day 14, at which time both Fas and Fas ligand presence were reduced by taurine supplementation.

FIG. 10C shows no effect of a low protein diet or taurine supplementation on iNOS, although a pronounced developmental increase was seen at postnatal day 12, just preceding the wave of apoptosis. Without being limited to theory, this suggests that while the timing of developmental apoptosis may be related to increased NO presence within islets, the amplitude of fetal and neonatal islet cell apoptosis may be more related to the Fas pathway which may be sensitive to LP diet and amenable to rescue by taurine.

FIG. 10D shows VEGF immunoreactivity in the pancreas decreases with a low protein diet, but taurine reverses the effect, regardless of developmental stage.

### Example 11

### Effect of Taurine Supplementation on Pancreatic Vascularization

Animals and diets were as described above in Example 1. The fetal pancreas was removed at day 21.5. Vascular density, expressed as a percent of area, and number of blood vessels per unit area were evaluated.

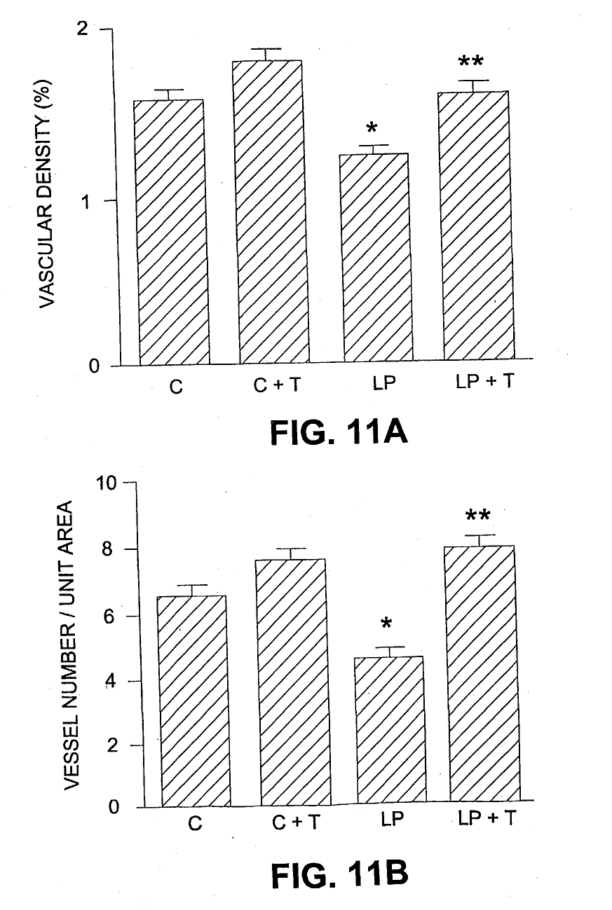

FIG. 11A and FIG. 11B show that vascular density and blood vessel numbers per unit area were reduced for the animals exposed to the maternal low protein diet. However, taurine supplementation in the drinking water reversed this effect, restoring both vascularization parameters to the level of the control groups. Statistically significant differences are as follows: (*) indicates a difference versus the (C) diet group (p<0.05), and ** indicates a difference versus the (LP) diet group (p<0.05).

### Example 12

### Interaction Effect of Dietary and in vitro Supplementation of Taurine

Animals and diets were prepared as described in Example 7. Pancreases were removed and islets were isolated from late gestation fetuses at day 21.5. Neoformed fetal islets obtained after five days of culture from the four diet groups (C, C+Taurine, LP, and LP+Taurine) were compared for their susceptibility to apoptosis following exposure to SNP (100 μmol/l), or IL1β (50 U/ml), with or without in vitro taurine at either physiological (0.3 mmol/l) or supraphysiological (3.0 mmol/l) levels.

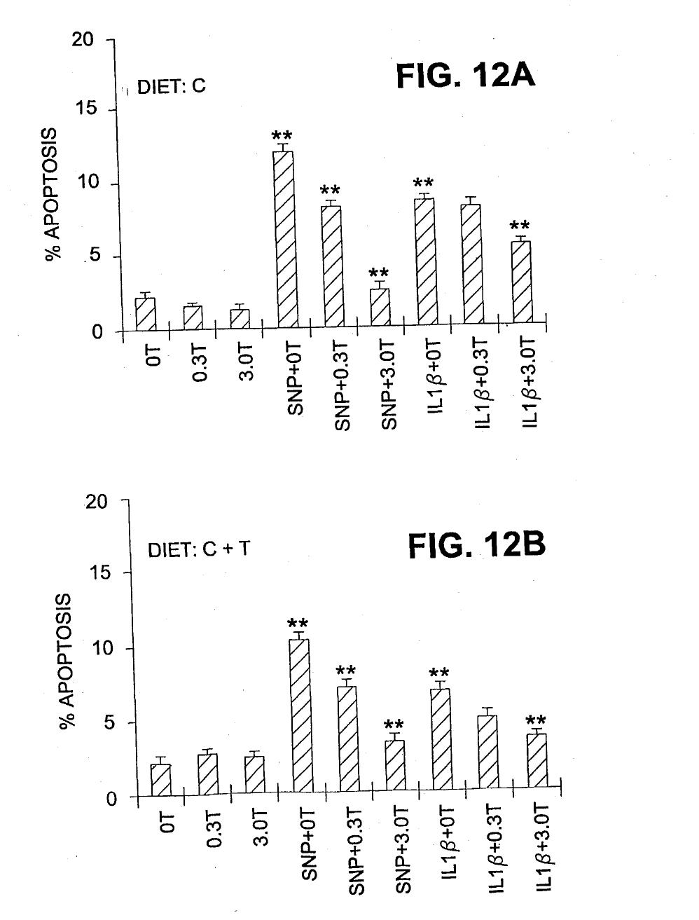

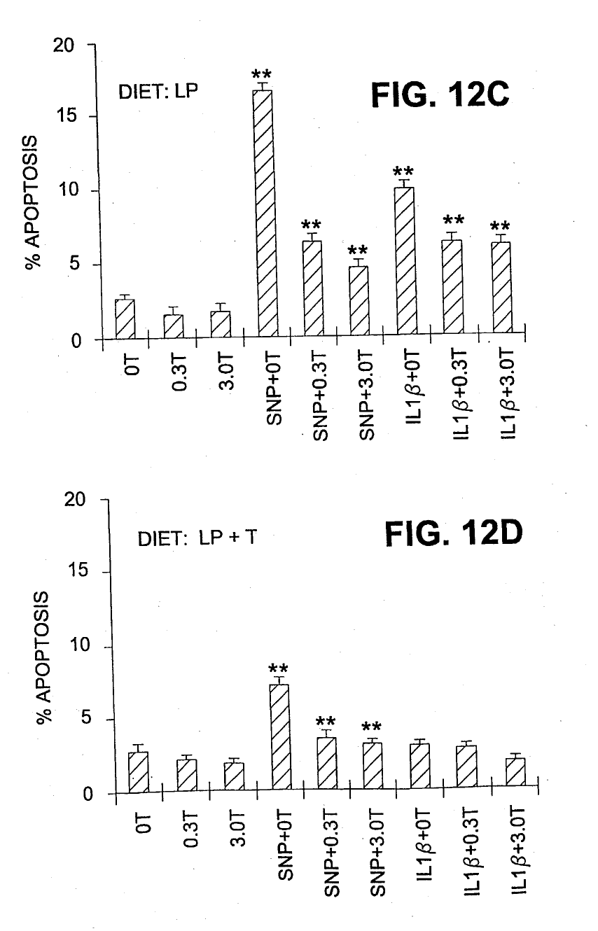

FIG. 12A to FIG. 12D illustrate the effect of maternal taurine supplementation on the sensitivity of fetal islets to apoptosis following in vitro exposure to SNP and IL 1β, in the presence of different levels of taurine. A statistically significant difference (p<0.01) from the control group without taurine is indicated by two asterisks (**).

FIG. 12A illustrates apoptotic rate for control animals (C), FIG. 12B shows apoptotic rate for control animals having supplemental taurine (C+Taurine), FIG. 12C shows apoptotic rate for low protein animals (LP), and FIG. 12D shows apoptotic rate for low protein animals receiving supplemental taurine (LP+Taurine). Compared to the control diet, fetal islets derived from the LP group showed an increased apoptotic response to SNP or IL1β. This was reversed by maternal supplementation with taurine in vivo, or co-incubation with taurine in vitro. These data indicate that a combination of dietary taurine supplementation in a low protein diet with a high local concentration of taurine in vitro synergistically decrease both SNP-induced and IL 1β-induced apoptosis in fetal islets.

### Example 13

Effect of Taurine on Incidence of Insulitis

Diabetes-prone non-obese diabetic (NOD) mice in which females develop a 90% rate of autoimmune diabetes by the age of 25 weeks were studied in order to determine the effect of taurine on insulitis onset and development. Insulitis initiates at 3-5 weeks of age in NOD mice, as leukocytes begin to infiltrate around ducts and venules in both female and male mice. These infiltrates progress toward the islets, which become surrounded by concentric layers of per-insular lymphocytes (non-destructive peri-insulitis). Destructive intra-islet insulitis then occurs, leading to extensive β cell destruction. All NOD mice display peri-insulitis, whereas intra-insulitis and overt Type 1 diabetes is restricted to about 70-80% of females and about 10-15% of males in the NOD mouse colony used in this instance. Insulitic infiltrates consist mainly of CD4⁺ and CD8⁺ T cells, but include some macrophages, B cells and natural killer (NK) cells.

The NOD mouse model of diabetes is a well established model directly comparable to human Type 1 diabetes. The NOD mouse spontaneously develops a disease closely resembling Type 1, diabetes in histology and range of autoimmune responses. Ultimately, the NOD mouse exhibits a loss of β cells in the pancreatic islets.

Pregnant NOD mice were maintained on a control diet either with or without taurine supplementation in the drinking water throughout pregnancy and lactation. Supplementation of taurine was stopped after weaning. At 12 weeks of age the animals were killed and examined for histological evidence of insulitis within the pancreatic islets. Mice which were examined and found to have evidence of insulitis, were then further scored as peri-islet (slight), less than 50% area of islet (medium) or more than 50% islet area (heavy), as indicative of the stage and/or severity of insulitis.

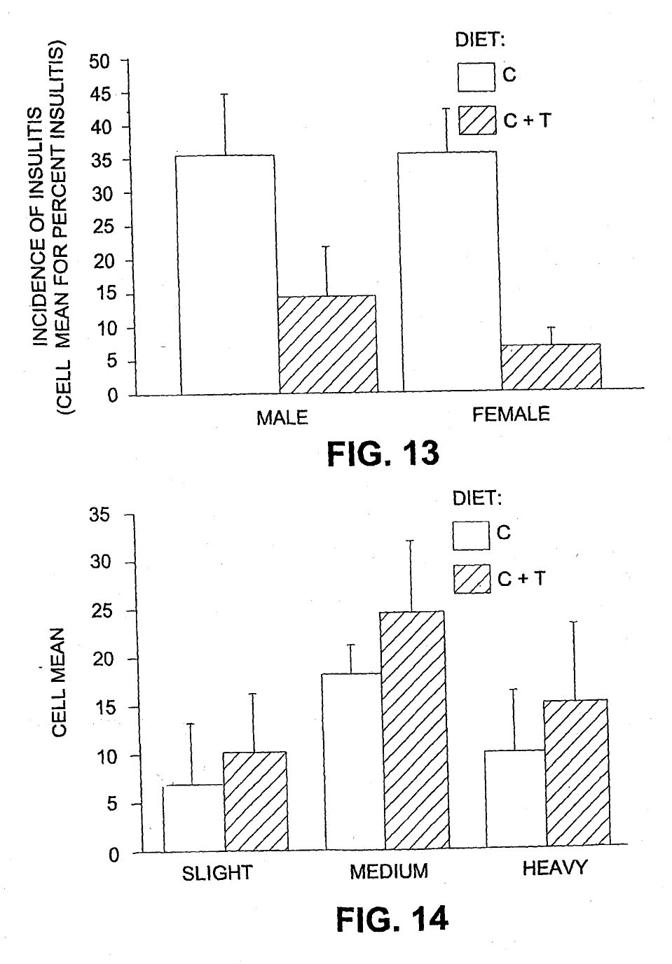

FIG. 13 illustrates that the incidence of insulitis was significantly reduced by 60% in male mice and 80% in female mice given taurine supplementation compared to control animals. Thus, taurine supplementation at the fetal and early post-natal stages of development reduced insulitis initiation. As insulitis is caused by autoimmune attack on pancreatic islets, a reduced incidence of insulitis is indicative of immunomodulatory activity.

FIG. 14 illustrates the severity of insulitis only in those female mice animals exhibiting insulitis. From the data of FIG. 13, it is clear that the incidence of insulitis is reduced. However, as illustrated here, the severity of the insulitis, when it does occur, is not lessened by taurine administration. FIG. 14 shows the severity of the insulitis in individual islets within female animals showing incidence of insulitis, scored as peri-islet (slight), less than 50% area of islet (medium) or more than 50% islet area (heavy). For the animals exhibiting insulitis, the proportion of individual islets showing no incidence of insulitis were approximately 50% in the control diet group receiving taurine supplementation (C+Taurine), and 65% in the control diet group (C). Taurine supplementation did not reduce the severity of the insulitis observed. This illustrates that although taurine limited the initiation of insulitis, as seen in FIG. 13, insulitis was not diminished by taurine once present.

### Example 14

### Sulfur-Containing Amino Acid Composition

A tablet form of a pharmaceutical composition for oral ingestion is prepared according to acceptable manufacturing practices. Each tablet comprises 1000 mg of taurine in combination with calcium carbonate as an inert diluent, and magnesium stearate as a lubricating agent. Five tablets are consumed per day by a human of 50 kg body weight.

### Example 15

### Maternal Taurine Supplementation Delays Onset of Diabetes in Offspring

Pregnant NOD mice were obtained and maintained on a control diet as described in Example 13. Pregnant mice were either supplemented with taurine in the drinking water (n=51) or were unsupplemented (n=37) throughout pregnancy. The supplemented group received taurine at a concentration of 2.5% (w/v) in drinking water. Considering the average amount of water consumed, this is equivalent to a taurine consumption of about 0.75 g/day per animal. Taurine supplementation ceased post-partum, and offspring were allowed to nurse for 21 days. Female offspring mice were observed for onset of diabetes up to 60 weeks post partum. Onset of diabetes was determined by the appearance of glucosurea which was assessed using glucose detection sticks.

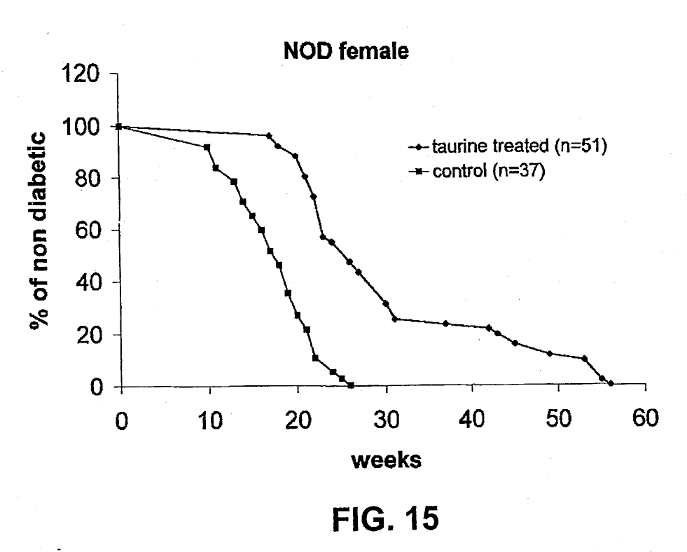

FIG. 15 illustrates the incidence of diabetes onset in the female offspring. Notably, the presence of taurine in the maternal drinking water impacted the rate of onset of diabetes in the offspring. At 25 weeks, almost all of the female offspring receiving no maternal taurine supplementation displayed diabetes. This is typical of the NOD mouse model for our colony. However, at 25 weeks, only about half of the female offspring receiving maternal taurine supplementation showed diabetes. At 50 weeks, long after the control group of offspring illustrated complete onset of diabetes, about 10% of the taurine-supplemented group still were not diabetic.

These data clearly illustrate that maternal taurine supplementation resulted in a remarkable delay of onset of islet dysfunction, in this case as shown by onset of diabetes, and increased survival of offspring prone to diabetes. This remarkable delay of diabetes onset can be attributed to the maternal supplementation, as there was no postpartum supplementation of any kind.

### Example 16

Gestational Taurine Supplementation Delays Onset of Autoimmune Diabetes in NOD Mice: Altered Development of the Endocrine Pancreas

The NOD mouse has an immune response in which insulitis precedes β-cell destruction. In this example, it was found that a reduced β cell mass induced by protein restriction during pregnancy is reversed by supplementation with 2.5% of taurine. The effects of taurine on the onset of diabetes in female NOD mice and on the development of the endocrine pancreas was studied.

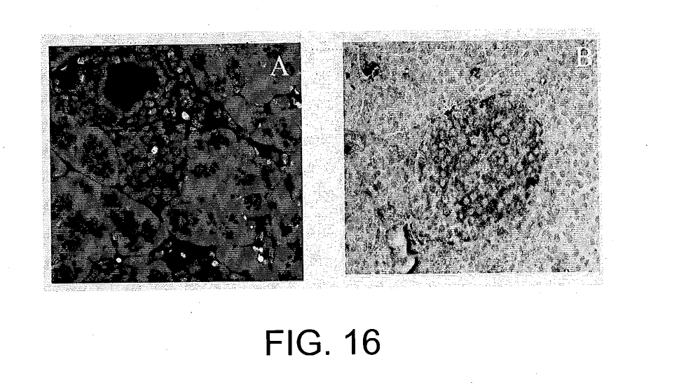

Pregnant NOD mice (maintained as in Example 13) were supplemented with 2.5% of taurine in drinking water during gestation. Pups were either sacrificed at 14 days of age, and pancreata were collected and fixed for histology as depicted in FIG. 16, or followed until glucosurea was apparent. Insulitis, which appeared at 8 weeks, was reduced by 90% by taurine supplementation.

FIG. 16 illustrates islet histology from 14 day old mice with and without gestational taurine supplementation.

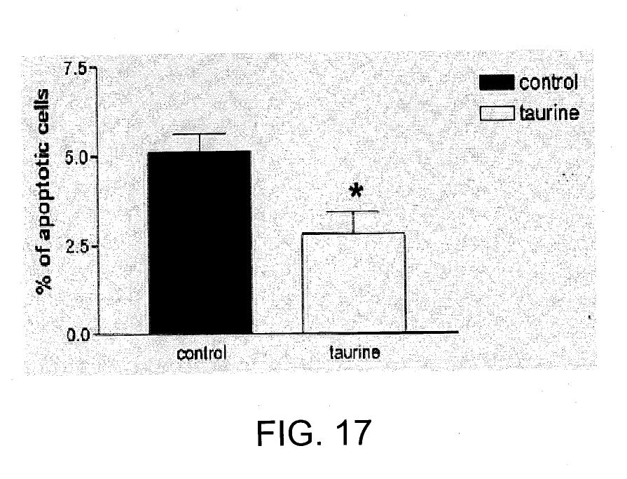

FIG. 17 illustrates apoptosis in islets from NOD female mice with and without gestational taurine supplementation. As noted above, taurine supplementation reduced nearly in half the number of apoptotic cells as compared to control animals.

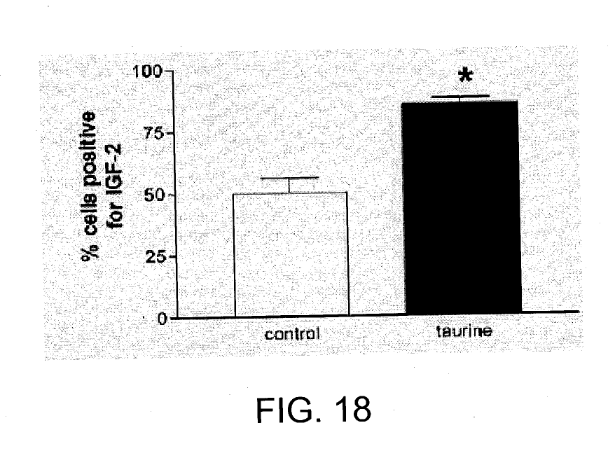

FIG. 18 illustrates the percentage of islet cells testing positive for IGF-2 immunoreactivity from NOD female mice with and without gestational taurine supplementation.

In controls, 50% of mice (n=37) became diabetic by 16 weeks of age, while taurine treatment postponed diabetes to 26 weeks (n=51) (p<0.001). At 14 days of age the mean islet area was significantly smaller in animals treated with taurine (p<0.05) due to a change in size distribution towards a greater number of smaller islets. Islets of taurine-treated mice also demonstrated a greater proportion of proliferating cells (p<0.05) as well as IGF-II-immunoreactive cells (p<0.01) (see FIG. 18) and had less apoptotic cells (p<0.01) as shown in FIG. 17. Thus, taurine-induced protection from diabetes involved an altered programming of endocrine pancreatic development.

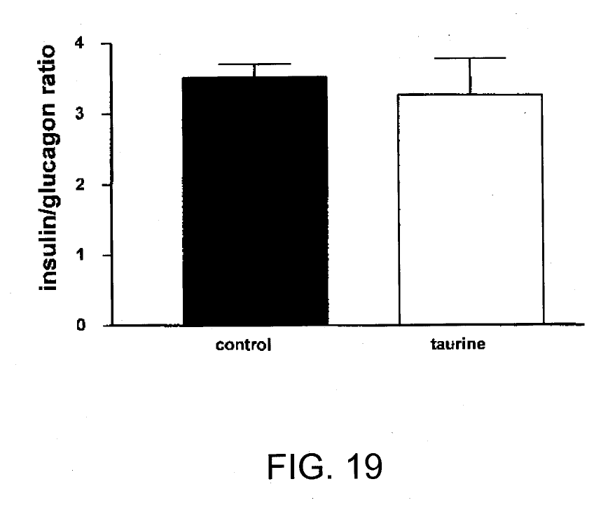

FIG. 19 shows the insulin/glucagon ratio for small islets in NOD female mice with and without gestational taurine supplementation. No difference was found in the relative ratio of insulin/glucagon ratio with or without taurine supplementation in small islets in NOD female mice.

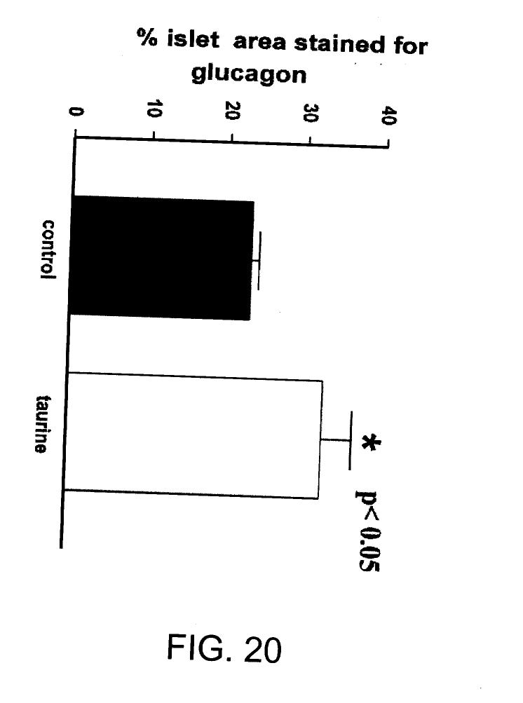

FIG. 20 shows the percentage of area stained for glucagon in small islets from NOD female mice with and without gestational taurine supplementation. Alpha cells were also significantly increased with taurine supplementation in small islets.

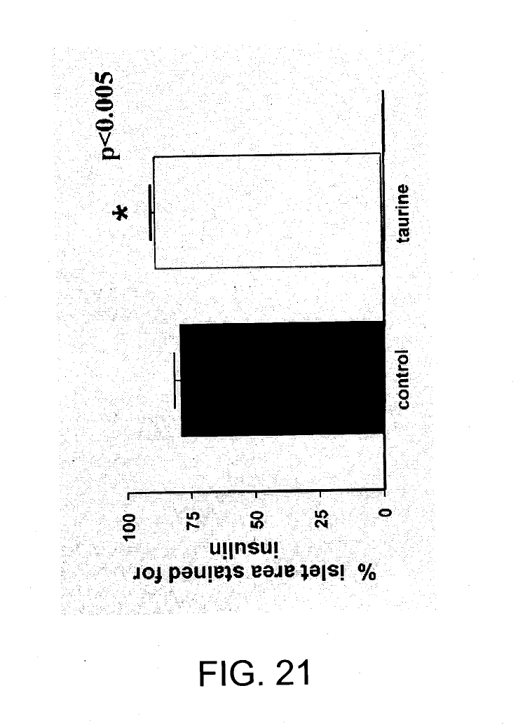

FIG. 21 shows the percentage of area stained for insulin in small islets from NOD female mice with and without gestational taurine supplementation. A significant increase was found in β mass in NOD female mice with taurine supplementation.

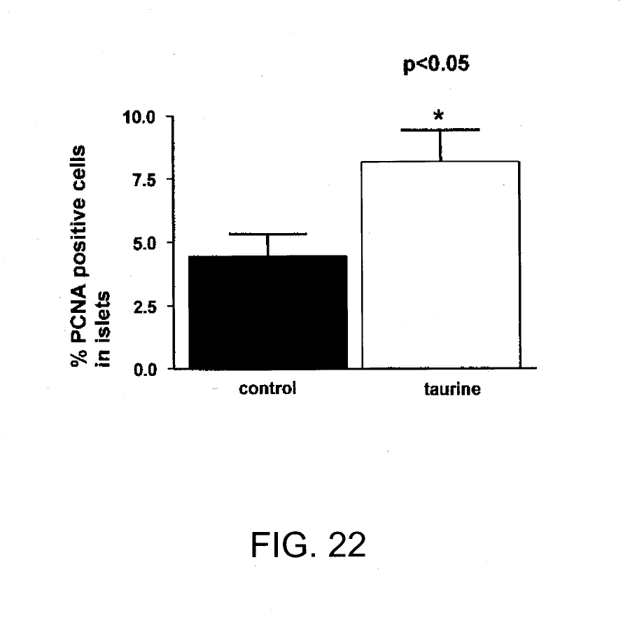

FIG. 22 shows the percentage of PCNA positive cells in small islets from NOD female mice with and without gestational taurine supplementation. A significant increase in cell proliferation was found in the small islets with taurine supplementation.

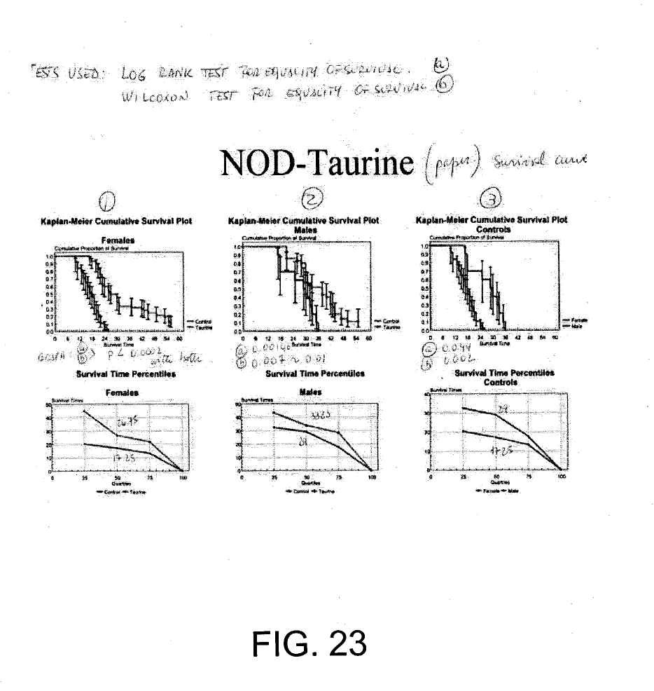

FIG. 23 illustrates survival plots from NOD mice with and without gestational taurine supplementation. Two different tests were used: (a) the Log Rank Test for Equality of Survival and (b) the Wilcoxon Test for Equality of Survival. Statistics were tabulated using GB-STAT software by Dynamic Microsystems Inc. (Silver Spring). Both female and male NOD mice showed improved survival with taurine supplementation.

The above-described embodiments of the invention are intended to be examples of the present invention. Alterations, modifications and variations may be effected the particular embodiments by those of skill in the art, without departing from the scope of the invention which is defined solely by the claims appended hereto. All references discussed above are herein incorporated by reference.

What is claimed is:

1. A composition for inhibiting islet dysfunction comprising an amino acid like structure carrying a sulfur moiety and a biologically acceptable carrier, wherein said amino acid like structure carrying a sulfur moiety is selected from the group consisting of taurine, L-cysteine, L-methionine, and combinations thereof.

2. The composition according to claim 1, wherein islet dysfunction comprises a condition selected from the group consisting of insulitis, Type 1 diabetes, Type 2 diabetes, mature onset diabetes of the young, and gestational diabetes.

3. The composition according to claim 1, wherein the amino acid like structure carrying a sulfur moiety exerts anti-apoptotic activity to inhibit islet dysfunction.

4. The composition according to claim 1, wherein the amino acid like structure carrying a sulfur moiety exerts immunomodulatory activity to inhibit islet dysfunction.

5. The composition according to claim 1, wherein islet dysfunction is inhibited in an offspring of a pregnant mammal.

6. The composition according to claim 1, wherein islet dysfunction is inhibited in a suckling offspring of a lactating mammal.

7. The composition according to claim 1, having a dosage form selected from the group consisting of a pharmaceutical preparation, a nutraceutical preparation, a functional food, a maternal supplement, and an infant formula.

8. A method of inhibiting islet dysfunction comprising administration of an effective amount of an amino acid like structure carrying a sulfur moiety to a mammal in need thereof, wherein said amino acid like structure carrying a sulfur moiety is selected from the group consisting of taurine, L-cysteine, L-methionine, and combinations thereof.

9. The method according to claim 8, wherein islet dysfunction comprises a condition selected from the group consisting of insulitis, Type 1 diabetes, Type 2 diabetes, mature onset diabetes of the young, and gestational diabetes.

10. The method according to claim 8 wherein the amino acid like structure carrying a sulfur moiety exerts anti-apoptotic activity to inhibit islet dysfunction.

11. The method according to claim 8, wherein the amino acid like structure carrying a sulfur moiety exerts immunomodulatory activity to inhibit islet dysfunction.

12. The method of claim 8, wherein said islet dysfunction is inhibited in an offspring of a pregnant mammal, said method comprising administration of an effective amount of an amino acid like structure carrying a sulfur moiety to the pregnant mammal.

13. The method of claim 8, wherein said islet dysfunction is inhibited in a suckling offspring of a lactating mammal, said method comprising administration of an effective amount of an amino acid like structure carrying a sulfur moiety to the lactating mammal.

14. A composition for inhibiting an autoimmune disorder comprising an amino acid like structure carrying a sulfur moiety and a biologically acceptable carrier, wherein said amino acid like structure carrying a sulfur moiety is selected from the group consisting of taurine, L-cysteine, L-methionine, and combinations thereof.

15. The composition according to claim 14, wherein said autoimmune disorder is selected from the group consisting of insulitis, Type I diabetes, rheumatoid arthritis, thyroiditis, and pancreatitis.

16. The composition according to claim 14, wherein said amino acid like structure carrying a sulfur moiety exerts anti-apoptotic activity to inhibit cell destruction resulting from said autoimmune disorder.

17. The composition according to claim 14, wherein said amino acid like structure carrying a sulfur moiety exerts anti-apoptotic activity to inhibit β cell destruction resulting from an autoimmune disorder selected from the group consisting of insulitis and Type I diabetes.

18. The composition according to claim 14, having a dosage form selected from the group consisting of a pharmaceutical preparation, a nutraceutical preparation, a functional food, a maternal supplement, and an infant formula.

19. The composition according to claim 14, wherein said autoimmune disorder is inhibited in an offspring of a pregnant mammal.

20. The composition according to claim 14, wherein said autoimmune disorder is inhibited in a suckling offspring of a lactating mammal.

21. A composition for delaying onset of diabetes in a mammal prone to diabetes, said composition comprising an amino acid like structure carrying a sulfur moiety and a biologically acceptable carrier, wherein said amino acid like structure carrying a sulfur moiety is selected from the group consisting of taurine, L-cysteine, L-methionine, and combinations thereof.

22. A method for delaying onset of diabetes in offspring of a pregnant mammal, comprising administration of an effective amount of an amino acid like structure carrying a sulfur moiety to said pregnant mammal, wherein said amino acid like structure carrying a sulfur moiety is selected from the group consisting of taurine, L-cysteine, L-methionine, and combinations thereof.
